(19) United States
(12) Patent Application Publication
(10) Pub. No.: US 2020/0411148 A1
(43) Pub. Date: Dec. 31, 2020

(54) Methods, Systems, and Mediums for Identifying Changes to the Brain Profile of an Individual

(71) Applicants: Board of Regents, University of Texas System, Austin, TX (US); Home Base Impairment Company, Inc., Coraopolis, PA (US)

(72) Inventors: Pamela J. Beehler, Mansfield, TX (US); George V. Kondraske, Arlington, TX (US); Michael A. Hauser, Moon Township, PA (US); Paul S. Chirgott, Coraopolis, PA (US); Mark A. Cavicchia, Coraopolis, PA (US); Clarence W. Carlos, II, Coraopolis, PA (US)

(21) Appl. No.: 16/727,418

(22) Filed: Dec. 26, 2019

Related U.S. Application Data
(63) Continuation of application No. 15/116,994, filed on Aug. 5, 2016, now abandoned.
(60) Provisional application No. 61/965,780, filed on Feb. 7, 2014.

(51) Int. Cl. G16H 15/00; G16H 50/30

(52) U.S. Cl. CPC G16H 15/00; G16H 50/30

(57) ABSTRACT

The invention relates to computer-based methods, systems, and mediums for identifying a change to the cumulative characterization of certain brain performance data collected from an individual, wherein the collected brain performance data is directly related to how at least one of the individual's neuromotor or cognitive brain functions is performing, hereinafter such a cumulative characterization is referred to as the individual's “Brain Profile”. The computer-based methods, systems, and mediums of the present invention identify and monitor changes in an individual's Brain Profile by employing of a computer system which is programmed and configured to perform at least the following tasks: to compute an individual's Normal Brain Profile through the implementation of a test session taken at least two separate times; to compute the individual's Current Brain Profile through the implementation the test session taken at a time after their Normal Brain Profile has been computed; and, to calculate whether there is a change to the individual's Brain Profile has occurred through a mathematical manipulation of their Normal Brain Profile and their Current Brain Profile.

### CROSS-REFERENCE TO RELATED APPLICATIONS

This application is a continuation of U.S. patent application Ser. No. 15/116,994, filed Aug. 5, 2016, which is the United States national phase of International Application No. PCT/US2015/014696 filed Feb. 6, 2015, which claims the benefit of U.S. Provisional Patent Application No. 61/965,780, filed Feb. 7, 2014, the disclosures of which are hereby incorporated in their entirety by reference.

### FIELD OF THE INVENTION

The present invention relates to methods, systems, and mediums for identifying changes to an individual's “Brain Profile”. While the term “Brain Profile” can be defined a number of different ways, as used herein, the term refers to the cumulative characterization of certain brain performance data collected from an individual, wherein the collected brain performance data is directly related to how at least one of the individual's neuromotor or cognitive brain functions is performing.

More particularly, the present invention relates to computer-based methods, systems, and mediums for identifying a change to an individual's Brain Profile, wherein the change is determined by employing a “computer system” (defined infra.) to perform at least the following tasks: to establish an individual's “Normal Brain Profile” (defined infra.) through the implementation of a test session taken at least two separate times; to establish the individual's “Current Brain Profile” (defined infra.) through the implementation of the test session taken at a time after their Normal Brain Profile has been established; and, to calculate whether there is a change to the individual's Brain Profile through a mathematical manipulation of their Normal Brain Profile and their Current Brain Profile, wherein any detected change can be the result of an impairment or enhancement to at least one of the individual's neuromotor or cognitive brain functions being tested.

### BACKGROUND OF THE INVENTION

The human brain is a soft organ made up of millions of nerve cells called neurons. These neurons form tracts that carry messages to different parts of the brain, which, in turn, control an individual's cognitive and neuromotor brain functions. The cumulative nature of an individual's neuromotor (e.g., eye-tracking, hand-eye coordination, etc.) and cognitive (e.g., verbal, iconic recall, etc.) brain functions at any given point in time constitutes that person's Brain Profile.

Notwithstanding the countless number of life-sustaining functions performed by the human brain on a daily basis, it is, none-the-less, fairly susceptible to having its functions easily altered which, in turn, will correlate to changes in an individual's Brain Profile. Alterations in a person's brain functions can be caused, for example, by changes to their brain resulting from injuries, diseases, psychiatric disorders, physical disorders, medication, substance abuse, food consumption, drink consumption, varying levels of sleep, and combinations thereof. This, in turn, can cause changes in that individual's Brain Profile.

As for brain injuries which can alter an individual's neuromotor and cognitive brain functions—and thus their Brain Profile, such can result from a bump, blow, or jolt to, or shaking of, the head, or by any other physical action or reaction that causes the head to move quickly back and forth or side to side (e.g., from a fall, a blast, an automobile accident, participation in a contact sport, etc.). An injury resulting from such a trauma is typically referred to in the medical profession as a concussion or a traumatic brain injury (TBI). In addition to concussions or TBIs, brain injuries can also result from a cumulative effect of multiple jolts to, or jarrings of, the head, even when of a much less severe nature.

In addition to injuries, changes to an individual's Brain Profile can result from a nervous system disease or disorder. Specifically, it is generally known in the medical profession that certain ailments of this nature can adversely affect a person's neuromotor or cognitive functions; and thus, result in a change to their Brain Profile. An example of some such presently-known ailments includes, without limitation, the following: Parkinson's Disease, Alzheimer's Disease, Amyotrophic Lateral Sclerosis (ALS)—often referred to as “Lou Gehrig's Disease”, and Huntington's Disease; as well as, all forms of dementia such as Vascular Dementia, Lewy Body Dementia, Frontotemporal Dementia, and HIV-associated Dementia.

A particular ailment that can cause changes to a one's Brain Profile, and that has also been associated with an individual experiencing multiple brain injuries such as concussions, TBIs, or “sub-concussions” (i.e., brain injuries which are less serious than concussions), is a type of dementia known as Chronic Traumatic Encephalopathy (CTE). CTE is a progressive degenerative brain disease that has been found in athletes participating in contact sports such as: football, baseball, and boxing. CTE has also been found in soldiers exposed to blast or concussive injuries. As technology advances, it is expected that CTE will also be discovered in many other activities wherein blows, jolts, or violent movement of the head are possible. An example of such other activities includes, without limitation, the following: hockey, rugby, lacrosse, gymnastics, cheerleading, martial arts, skiing, and automobile, motorcycle, bicycle or boat racing. CTE and other closed cranial injuries may also be suffered during a vehicle accident, a fall, a battery, a trauma suffered at birth, or any event during the course of life where an external force is directly or indirectly applied to the cranium.

Individuals with concussive and closed cranial injuries, such CTE, TBIs and sub-concussions, may experience neurological, structural, muscular, and/or organic impairment. If experienced, these impairments may occur immediately after the injury, or may take months, years, or even decades, to manifest themselves.

Depending upon the severity of the injury, the neurological impairment that one may experience includes impairment to the brain, nerves, and/or sensory organs. For example, the sensory organs, particularly those residing in the head, may be affected by a cranial injury, as well as the nerve pathways that link those sensory organs to the brain.

Examples of structural impairments that can result in changes to one's Brain Profile include impairments to that person's cranium and meninges. Specifically, when the head is struck by either an object or a concussive force, the cranial bones displace to absorb the shock and protect the brain. This displacement typically does not self-correct; and in their displaced condition, the cranial bones no longer move as they should. Because of the very close tolerances within the skull, their displacement also negatively affects the meninges, adjacent brain tissue, general and localized blood flow to the brain, and cerebrospinal fluid (CFS) flow.

Examples of muscular impairments that can result in changes to one's Brain profile include impairments to that person's ocular and facial muscles. The ocular and facial muscles all attach to cranial bones. When intrinsic movement of these bones is compromised, the muscles attached to them are either stretched or compressed. This, in turn, may result in them being unable to function properly.

Examples of organic impairments that can result in changes to one's Brain Profile include impairment resulting from the lack of oxygenation, lack of nourishment, and impaired and/or reduced metabolic waste removal to or from that person's brain. For example, in some instances, it has been shown that cranial bone movement may results in two conditions that affect the brain's ability to function properly—ischemia (restricted blood supply) and reduced CSF. Specifically, if a particular brain injury results in ischemia, this, in turn, results in the reduction of oxygen and nutrient levels supplied to the brain. Over time, these two conditions progressively reduce the brain's ability to perform a wide range of functions.

In addition to the above, one's Brain Profile can change by any one or more of the following functions becoming compromised or impaired as a result of a cranial injury: neuromotor functions, ocular functions, visual perceptions, and other functions attributed to the left or right side of the brain. As technology advances, numerous other impairments to the human body associated to injuries to the brain will undoubtedly be discovered. All such presently known, and future discovered, impairments can, in turn, result in changes to an individual's Brain Profile.

Non-nervous system diseases can also cause impairments to one's neuromotor and cognitive functions; and thus, result in changes to their Brain Profile. For example, diabetes has been known to cause blurry vision, fatigue, and mental confusion. These symptoms, in turn, can interfere with one's cognition and ocular functions.

One's Brain Profile can also be changed by physical and psychiatric disorders. For example, physical disorders such as epilepsy have been known to manifest symptoms including unpredictable seizures. These, in turn, interfere with coordination and/or control of muscles throughout the body.

Examples of psychiatric disorders that can result in a change to one's Brain Profile include disorders such as: depression, anxiety disorders (e.g., panic disorders, obsessive compulsive disorders, post-traumatic stress disorders, social anxiety disorders, phobias, etc.), bipolar disorders, attention deficit disorder (e.g., ADHD, ADD, etc.), and schizophrenia. Since these disorders have been known to manifest symptoms including difficulty in concentrating, fatigue, hyperactivity, memory loss, other cognitive impairment, and/or loss of motivation, they have also been known to interfere with coordination and/or control of muscles throughout the body.

In addition to injuries, diseases, and ailments to and of the brain, changes to one's Brain Profile can also result from what that person consumes, inhales, or injects into their body. For example, while some medications are designed to improve or enhance a person's neuromotor or cognitive brain functions, these same medications can produce deleterious effects. Similar enhancements or impairments to an individual's neuromotor or cognitive functions can also result from their ingestion, inhalation, and/or injection of narcotic or controlled substances, as well as from the consumption of alcoholic or other intoxicating beverages.

Substance use or abuse that can alter one's Brain Profile includes various uses, overuses, and misuses of prescription drugs, non-prescription drugs, illicit drugs (e.g., methamphetamines, cocaine, etc.), legalized intoxicants (e.g., alcohol), and/or unregulated substances (e.g., caffeine pills, energy drinks, etc.). It has been shown that such substance use or abuse can impact the normal function of one's central nervous system. For example, alcohol or drug abuse can impair eye movement by slowing the function of the central nervous system and interfering with coordination of muscles throughout the body.

Moreover, changes to one's Brain Profile can also result from the amount of sleep that they are, or are not, getting. For example, insomnia and sleep deprivation has been reported to produce some of the same deleterious effects on one's neuromotor and/or cognitive brain functions, as is experienced with substance or alcohol abuse.

Other conditions which can impact one's Brain Profile include aging, fatigue, stress, and pain. For example, fatigue due to physical exertion or to lack of sleep, has been shown to impair eye movement by slowing the function of the central nervous system and interfering with cognition and coordination of muscles throughout the body.

Accidental or naturally-occurring events can also change one's Brain Profile. Examples of such events include the accidental or occupational exposure to toxic chemicals. These events can damage the central nervous system and, as above, interfere with cognition and coordination of muscles throughout the body.

In addition to identifying impairments which result in the change in one's Brain Profile, it is also often times equally important to identify enhancements which result in a change to the same. For example, if a person is on a particular treatment or rehabilitation regime, it is extremely helpful to know early on in that process as to whether, and/or to what extent, the chosen process is actually working. If the injury, disease, disorder or ailment being treated also adversely effects one of the patient's brain functions, by monitoring changes to their Brain Profile, the present invention can be used as a tool to determine the effectiveness of the chosen treatment or rehabilitation regime.

As for many diseases, ailments, disorders, or injuries of the brain, early detection is often times very instrumental in treating the same, and/or in preventing further damage from occurring. For example, early detection of a particular disease, ailment, disorder, or injury can offer the individual, a parent, a guardian, a caregiver, or some other third party, with the opportunity to intervene medically. This early intervention, in turn, can result in a better chance for a cure or rehabilitation, as well as in some instances, for the saving of lives. Such an early detection is now possible by monitoring changes to one's Brain Profile.

Early detection of changes to one's Brain Profile can also be very important when applied to those individuals who work in occupations where the foreknowledge of one having unimpaired or unenhanced brain functions is extremely desirable, if not entirely necessary. An example of current occupations where such knowledge is important includes, without limitation, the following: aircraft/spacecraft crew, air traffic controllers, watercraft operators, truck drivers, train engineers, mass transit drivers, military personnel, law enforcement officers, and security guards.

While the prior art is replete with numerous commercialized, computer-based neurocognitive testing methods (hereinafter referred to as the “prior art testing methodologies”), most of them are directed towards diagnosing whether an individual has suffered a TBI or a concussion. As such, most these prior art testing methodologies focus on evaluating only those specific brain functions which are supposedly altered when an individual encounters some sort of a concussive brain injury. Examples of such prior art testing methodologies include the following: Immediate Postconcussion Assessment and Cognitive Testing (ImPACT), Axon Sport's Computerized Cognitive Assessment Tool (SCCAT), Cleveland Clinic Concussion System (C3), U.S. Military's Automated Neuropsychological Assessment Metrics (ANAM), Neurobehavioral Evaluation Systems (NES), and Headminder's Concussion Resolution Index (CRI).

Certain of the aforementioned prior art testing methodologies have been used for, and by, athletes at the K-12, collegiate, and professional levels. When these prior art testing methodologies are employed, the athlete is typically assessed once, while supposedly healthy and unimpaired, in order to create for them single-point baseline neuropsychological scores. If employed, the athlete's single-point baseline scores are collected either once a season, or once a year, or even sometimes, once every multiple years. After being collected, when someone (i.e., the athlete, their parent, a coach, a trainer, or some other third party) believes that trauma to the head of that athlete has occurred, in response to such a belief, the athlete is tested again to see how their subsequent neuropsychological scores compare to their single-point baseline scores. If there is no substantial difference in the two scores, it is typically concluded that no concussive injury has occurred.

As mentioned above, most of the prior art testing methodologies are directed for use by contact-sport athletes. Accordingly, those who are not athletes, and/or who do not have available to them these current testing methodologies, are left to more conventional methods of diagnosing concussive brain injuries, such as going to an Emergency Room and/or seeking the attention of a medical professional.

Also as mentioned, most of the prior art testing methodologies focus their tests on those specific brain functions which are altered when the athlete encounters a concussive brain injury. Accordingly, these testing methodologies may not be able to assist anyone, including athletes, whose brain functions are altered for reasons other than concussive brain injuries (e.g., alterations due to certain brain diseases or ailments, certain psychiatric or physical disorders, alcohol or substance abuse, sleep deprivation, stress, fatigue, pain, etc.). Rather, the observation of such brain function altering influences typically requires the use of sophisticated medical instrumentation, as well as generally taking hours, or days, to administer, evaluate, and report results. By the time such an evaluation and reporting occurs, the particular malady can have significantly worsened and may no longer be curable or reversible.

Moreover, most the prior art testing methodologies are “reactive” in nature. In other words, they are generally not employed or administered until the athlete, or some other third party, believes that the athlete has encountered some sort of a head trauma. This adds a fairly substantial subjective component to the existing testing methodologies which, in turn, can result in the lesser severe injuries going undetected until they worsen and become much more substantial.

In addition to their reactive and subjective natures, most the prior art testing methodologies need to be administered and/or evaluated by some sort of a trained professional. This can cause scheduling problems which, in turn, can result in it taking days, or even weeks, before the individual gets evaluated and diagnosed.

Since many changes to the brain function(s) making up an individual's Brain Profile typically start at a relatively small level and, often times, progress slowly over time, with the prior art testing methodologies, such changes are often difficult to detect and/or observe until they become much more severe. Notwithstanding the clear advantages associated with the immediate and early detection of impairments or enhancements to an individual's brain functions (including those individuals who are not contact-sport athletes), at the present time, there is no proactive, simple to take, cumulative, and objective method of doing the same.

Accordingly, it is one object of this invention is to provide proactive, simple to take, cumulative, and objective methods, systems, and mediums for identifying changes to an individual's Brain Profile, including those individuals who are not contact-sport athletes. Other objects will become apparent to those skilled in the art as the invention becomes better understood by reference to the following Detailed Description, especially when considered together with the accompanying Claims appended hereto, and the Figures, briefly described below.

### SUMMARY OF THE INVENTION

Briefly, the present invention is directed to providing proactive, simple to take, cumulative, and objective methods, systems, and mediums for identifying a change to an individual's Brain Profile, including those individuals who are not contact-sport athletes.

In accordance with various embodiments, the present invention comprises computer-based methods, systems, and mediums for identifying a change to an individual's Brain Profile, wherein the change is determined by employing a “computer system” (defined infra.) to perform at least the following tasks: to establish an individual's “Normal Brain Profile” (defined infra.) through the implementation of a test session taken at least two separate times; to establish the individual's “Current Brain Profile” (defined infra.) through the implementation the test session taken at a time after their Normal Brain Profile has been established; and, to calculate whether there is a change to the individual's Brain Profile through a mathematical manipulation of their Normal Brain Profile and their Current Brain Profile, wherein any detected change can be the result of an impairment or enhancement to at least one of the individual's neuromotor or cognitive brain functions being tested.

In accordance with further embodiments, the present invention comprises computer-readable tangible non-transitory medium storing instructions for identifying a change to an individual's Brain Profile, wherein the change is determined by employing a “computer system” (defined infra.) to perform at least the following tasks: to establish an individual's “Normal Brain Profile” (defined infra.) through the implementation of a test session taken at least two separate times; to establish the individual's “Current Brain Profile” (defined infra.) through the implementation of the test session taken at a time after their Normal Brain Profile has been established; and, to calculate whether there is a change to the individual's Brain Profile through a mathematical manipulation of their Normal Brain Profile and their Current Brain Profile, wherein any detected change can be the result of an impairment or enhancement to at least one of the individual's neuromotor or cognitive brain functions being tested.

A more complete appreciation of the present invention, and many of the attendant advantages thereof, will be readily ascertained by and to those skilled in the art as the invention becomes better understood by reference to the following Detailed Description, especially when considered together with the accompanying Claims appended hereto, and the Figures, briefly described below.

### BRIEF DESCRIPTION OF THE DRAWINGS

The present invention is illustrated by way of example, and not by way of limitation, in the figures of the accompanying drawings, in which:

FIG. 1 is a flow diagram illustrating a system of one particular embodiment of the present invention, wherein a computer system is employed to determine whether there is a change to the individual's Brain Profile.

FIG. 2 is a flow diagram illustrating a system of another particular embodiment of the present invention, wherein a computer system is employed to determine whether there is a change to the individual's Brain Profile; and wherein the individual's Normal Brain Profile is computed after a “normal” score (defined infra.) has been established for each test module making up a particular test session.

FIG. 3 is a flow diagram illustrating a system of yet another particular embodiment of the present invention, wherein a computer system is employed to determine whether there is a change, not only to the individual's Brain Profile; but also, to the individual's separate test module scores making up a particular test session.

FIG. 4 is a table illustrating a particular set of test modules making up an illustrative example of a particular test session; as well as, the grouping of those modules within the test session.

### DETAILED DESCRIPTION

Unless defined otherwise, all technical, medical, and scientific terms used herein have the same meanings as commonly understood by those of ordinary skill in the art. While specific methods, devices, mathematical computations, systems, and mediums are being described, any methods, devices, mathematical computations, systems, and mediums that are similar or equivalent to those described herein can be used in the practice of, and are encompassed by, the present invention. This includes such similar or equivalent methods, devices, mathematical computations, systems, and mediums, which have not yet been discovered or developed.

In the following description, details are set forth in order to provide a more thorough understanding of the present invention. That being said, it will be apparent to skilled artisans that the various embodiments of the invention disclosed herein may be practiced without all of the recited specific details.

The present invention pertains to proactive, simple to take, cumulative, and objective methods, systems, and mediums for immediately identifying changes to the Brain Profile of an individual through the use of a computer system. Most of the prior art methodologies used for measuring certain brain functions are “reactive” in nature; because, they are typically not employed until either the individual, or some other third party, believes there to be a problem resulting in that individual's brain function(s) being altered. If a brain injury is not caused by an obvious traumatic event—such as an automobile accident or a crushing blow to the head during a sporting event—the underlying problem is often times difficult to observe and diagnose until it, or its associated symptoms, become(s) much more severe.

Even in incidents where a traumatic event occurs, the diagnosis of a potential problem using the prior art testing methodologies still requires the “subjective” evaluation of the same, either by the injured individual, or by some other third party. Therefore, when such current prior art testing methodologies are employed, the correct diagnosis of a possible brain function-altering malady is largely dependent upon, not only the problem being readily apparent; but also, the problem's apparentness actually being observed by the individual, or by the some other third party. If either one of the aforementioned subjective criteria is not present, the problem, again, can go undetected until it, or its associated symptoms, become(s) much more severe.

Also, in the current prior art testing methodologies, the administration of the same typically has associated therewith the following characteristics: they are relatively long processes (e.g., they generally take between 1 to 4 hours to administer); they are only administered at a limited number of designated locations (e.g., at a doctor's office, at a clinic, at a hospital, in a trainer's office, etc.); and, they are administered and evaluated by a trained professional (e.g., an adequately trained M.D., Ph.D., physician's assistant, nurse practitioner, nurse, sport's trainer, etc.). These inherent characteristics of the prior art testing methodologies generally make processes designed to evaluate and assess changes in one's brain function(s) much less readily available to the general population of individuals who wish the same to be monitored and tested on a fairly periodic basis.

In addition to the foregoing, in most of the prior art testing methodologies, once a potentially injured individual has been able to schedule a time and place in which to be tested, and once the testing has been completed, the evaluation of the collected brain performance data is: (a) performed by a trained professional, and (b) not instantaneous. As such, when using the current prior art testing methodologies, after the test has been administered, the time necessary to evaluate and report the results can range from an additional several hours to an additional several days.

The aforementioned inherent characteristics of prior art testing methodologies make them relatively difficult and cumbersome to take, administer, and evaluate. This results in them being employed much less frequently (e.g., seasonally, annually, or multi-annually), which, in turn, can result in the early stages of an underlying brain function-altering malady going undetected. However, as will be demonstrated below, the methodologies of the present invention resolve all of the aforementioned deficiencies associated with those in the prior art.

Specifically, as mentioned above, one of the differences between the methodologies of the present invention and those of the prior art is that the present invention methodologies are “proactive” as opposed to “reactive” in nature. In other words, with the present invention, the test is designed to be taken on a relatively continuous basis (e.g., daily, weekly, or monthly), regardless of whether anyone believes that the individual has encountered some sort of a brain function-altering malady. As such, the methodologies of the present invention work to proactively detect the onset of potential problems to the brain. This, in turn, empowers the taker, their parent, their guardian, their caregiver, or some other third party, to intervene much earlier than was ever before possible.

Another difference between the methodologies of the present invention and those of the prior art is that the present invention methodologies are “objective” as opposed to “subjective” in nature. In other words, with the present invention, the test is not taken only when the taker, their parent, their guardian, their caregiver, or some other third party subjectively believes that something has occurred which is changing the brain function(s) making up an individual's Brain Profile. Rather, since the tests encompassed by the present invention are designed to be taken on a relatively continuous basis, they do not require any subjective evaluation prior to them being taken. This feature, in turn, removes the problems associated with a test not being taken because of an improper or incomplete diagnosis or evaluation by the individual, their parent, their guardian, their caregiver, or some other third party.

Yet another difference between the methodologies of the present invention and those of the prior art is that the present invention methodologies provide a cumulative test session scores. Specifically, the methodologies of the present invention not only employ a test session which comprises at least two separate and distinct test modules; but also, collects brain performance data from each test module and computes therefrom a single, cumulative test session score through a mathematical manipulation of the collected brain performance data. As used herein, the term “brain performance data” refers to a user's inputted responses to a test module's inquiries. After reading this description, numerous computation methods that can be employed when practicing this invention will become apparent to those skilled in the art. All such methods, be they past, present or future, are deemed to be encompassed by the present invention

Still a further difference between the methodologies of the present invention and those of the prior art is that the present invention methodologies are relatively simple to take. Specifically, unlike the prior art testing methodologies, those of the present invention generally take less than 20 minutes, and often times less than 15 minutes to, not only complete; but also, receive a comprehensive response as to whether there is a change in the taker's Brain Profile. Also adding to the simplicity of the present invention methodologies is the fact that they can be administered by anyone, even the taker him or herself (i.e., with the present invention, there is no need of any third party to explain, monitor, and/or evaluate the test, or its results). Moreover, unlike the current prior art methodologies, those of the present invention can be taken anywhere that the taker can access the invention's computer system server device via a communications network (defined infra). An example of such places include, without limitation, a classroom, a parked vehicle, the taker's own home, or anywhere else that a Wi-Fi or internet connection is available and accessible. These aforementioned features facilitate the probability that the tests of the present invention will be taken on a fairly frequent and consistent basis, which, in turn, facilitate the probability that relatively small changes in one's Brain Profile will be detected, and that the underlying malady can be identified at a much earlier stage.

In view of the above, practicing the methodologies of the present invention now empowers an individual, their parent, their guardian, their caregiver, or some other third party reviewing the results, to quickly and easily detect changes to one's Brain Profile. The ability to detect such changes offers individuals the opportunity to early intervene with a remedial or curative treatment plan. Early intervention, in turn, often times results in an increased and quicker chance of a cure or repair of the underlying problem.

Notwithstanding the aforementioned advantages of the present invention, it goes much further than merely providing a means for the proactive, simple, and objective identification of possible “impairments” to one's Brain Profile. Specifically, since the current invention results in a relatively continuous monitoring process, it is also a means for the proactive, simple, and objective identification of possible “improvements” or “enhancements” to one's Brain Profile. This latter feature can be very advantageous for a number of reasons. For example, if any individual (e.g., a child, athlete, non-athlete, adult, senior citizen, etc.) encountered some sort of a concussion or TBI, the implementation of the present invention will provide a quick, simple, cumulative, and objective indication of, not only how and to what extent the individual's Brain Profile was altered; but also, how and to what extent their Brain Profile is improving during the prescribed recuperative process. The present invention can also be used as a means of detecting when one's Brain Profile returns, if ever, to its pre-malady status.

In addition to the above, most current prior art testing methodologies are mainly directed towards detecting problems associated with concussive brain injuries. While the methodologies of the present invention can be used for such purposes, they can also be used to detect other types of problems or maladies that can result in one's Brain Profile changing. Examples of such include, without limitation, the following: those resulting from brain diseases and ailments, sleep deprivation, drug use and abuse, and alcohol consumption.

As mentioned above, due to the proactive, simplistic, cumulative, and objective natures of the methodologies encompassed by the present invention, it is very easy for them to be taken on a relatively frequent and continuous basis. The more times the methodologies of the present invention are taken, the more accurate they become in detecting minor changes to one's Brain Profile.

When practicing this invention, prior to determining whether there is a change in a user's Brain Profile, the computer system must first establish the user's “Normal Brain Profile” (defined infra). Thereafter, the computer system is programmed and configured to determine whether a change has occurred to the user's Brain Profile by comparing the user's earlier-computed Normal Brain Profile to one of their subsequently-computed “Current Brain Profiles” (defined infra). After reading this description, numerous computation methods that can be employed for making this calculation will become apparent to those skilled in the art. All such methods, be they past, present or future, are deemed to be encompassed by the present invention.

As used herein, the term “Normal Brain Profile” refers to a “consistent range” (defined infra) of brain performance data which represents the normal (i.e., typical and predominantly non-impaired or non-enhanced) performance, for that particular individual, of their at least one brain function being tested. This consistent range is calculated from at least two of the user's earlier-computed Brain Profiles, provided that these Brain Profiles are ones that qualify for use in making such a calculation—hereinafter, a “Qualifying Brain Profile”. If, however, the Brain Profiles are ones that do not qualify for use in making such a calculation, those particular Brain Profiles are hereinafter referred to as “Non-Qualifying Brain Profiles”. After reading this description, numerous computation methods that can be employed for making this calculation will become apparent to those skilled in the art. All such methods, be they past, present or future, are deemed to be encompassed by the present invention.

In accordance with the present invention, in order for a computed Brain Profile to qualify for use in the calculation of one's Normal Brain Profile, the Brain Profile must, itself, be computed from the scores of “Qualifying Test Modules”. As used herein, the term “Qualifying Test Modules” refers to “Test Module Scores” (defined infra) that was computed from collected brain performance data which fell within a certain pre-determined set of parameters. If, however, the Test Module Scores were computed from collected brain performance data which fell outside of a certain pre-determined set of parameters, those particular test modules are hereinafter referred to as “Non-Qualifying Test Modules”. The determination of what constitutes the aforementioned parameters is largely dependent upon the function being tested, the manner in which the function is being tested, and/or the needs and requirements of the end user. As described herein, a “Test Module Score” is a cumulative score resulting from a mathematical manipulation of the user's brain performance data collected upon the completion of each test module making up a particular test session.

That being said, in one embodiment, the set of parameters employed to determine when the module is characterized as a Qualifying Test Module is dependent upon the maximum human limit of the brain function(s) being tests by that module. Specifically, while there will always be some differences with regards to the maximum levels at which particular brain functions perform, there is an upper limit to those levels. This upper lever is hereinafter referred to as the function's “Maximum Human Capacity”. Upon knowing how a brain function is requested to perform within a particular test module, the Maximum Human Capacity limit for that brain function can be determined. Once determined, the Maximum Human Capacity can be multiplied by a factor of “X” to set the upper limit of that function's qualifying parameter; and by a factor of “Y” to set the lower limit of that function's qualifying parameter. The range of qualifying brain performance data is hereinafter referred to as that function's “Capacity Index”. The factor of “X” used in the aforementioned calculation can be a number which is less than, equal to, or greater than, one. Similarly, the factor of “Y” used in the aforementioned calculation can also be a number which is less than, equal to, or greater than, one. The setting of a brain function's Capacity Index for a particular test module (i.e., the selection of the appropriate “X” and “Y” factors) is largely dependent upon the function being tested, the manner in which the function is being tested, and/or the needs and requirements of the end user.

As the number of earlier-computed Qualifying Brain Profiles used in the Normal Brain Profile-calculation increases, so does the accuracy of the resulting computation. Accordingly, while the present invention requires that at least 2 earlier-computed Qualifying Brain Profiles are used in the Normal Brain Profile-calculation, preferably at least 10; more preferably, at least 20; and even more preferably, at least 30 of the user's earlier-computed Qualifying Brain Profiles will be used in such a calculation. After reading this description, numerous computation methods that can be employed for making this calculation will become apparent to those skilled in the art. All such methods, be they past, present or future, are deemed to be encompassed by the present invention.

When used herein, the term “Normal”, as it pertains to a user's “Normal Brain Profile” (defined supra) or their “Normal Test Module Scores” (defined infra), is not intended to mean that they fall within a predefined parameter which is established by a cumulative analysis of scores from multiple individuals within a particular population or group; or that there is absolutely no impairment or enhancement associated therewith. Rather, it merely means that, for a specific, predominantly non-impaired and non-enhanced individual, the scores making up the same fall within an earlier-computed “consistent range” for that parameter.

In accordance with this invention, the aforementioned “consistent range” is user specific. This is an important calculation being made and considered by the present invention since a set of scores which fall within the normal range for one individual, may not be within the normal range for another. For example, the consistent range of scores making up the Normal Brain Profile for a first, unimpaired individual may varying by a factor of “±1×”, while the consistent range of scores making up the Normal Brain Profile for a second, unimpaired individual may varying by a factor of “±2×”. If after their Normal Brain Profiles are established, both of these individuals complete a subsequent test session, and if both of the resulting Current Brain Profiles vary by a factor of “±1.5×”, the present invention will report that only the Brain Profile of the first individual has changed (i.e., the subsequently-computed Current Brain Profile score of ±1.5× falls outside of the first user's Normal Brain Profile; but, within the second user's Normal Brain Profile).

When calculating the consistent range making up the user's Normal Brain Profile, the computer system is programmed and configured to first compute a user-specific baseline from the scores of at least two of the user's earlier-computed Qualifying Brain Profiles. After the aforementioned baseline is computed, the computer system is then programmed and configured to compute what constitutes the consistent range of scores which are necessary to establish the user's Normal Brain Profile. When computing the consistent range of scores, it is within the scope of one embodiment of this invention for the computer system to be programmed to consider those test session scores used in the baseline calculation. However, it is also within the scope of another embodiment of this invention for the computer system to be programmed to ignore those test session scores used in the baseline calculation. Here, the test session scores used in the consistent range calculation would be those which are immediately collected after the baseline has been calculated. Moreover, it is further within the scope of yet another embodiment of this invention for the computer system to be programmed to consider only a portion of those test session scores used in the baseline calculation, in addition to those which are immediately collected after the baseline has been calculated. After reading this description, numerous computation methods that can be employed for making this calculation will become apparent to those skilled in the art. All such methods, be they past, present or future, are deemed to be encompassed by the present invention.

Once an individual's uniquely specific Normal Brain Profile is established, every subsequent time that a test session is completed, the resulting Brain Profile will be that which represents the performance of the individual's tested brain function at that current point in time—hereinafter referred to as the user's “Current Brain Profile”. To insure consistency and accuracy of results, the individual's Current Brain Profile should be based upon the same test session that was employed when their Normal Brain Profile was established. It is important to note, however, that while the phrase “the same test session” encompasses subsequent test sessions wherein neither the order of the test modules, nor the inquiries therein, are randomized, it is not limited to such a definition. Specifically, the phrase “the same test session” also encompasses subsequent test sessions wherein: (a) only the order of the test modules is randomized; (b) only the inquiries within the particular test modules are randomized; or (c) both, the order of the test modules and the inquiries therein, are randomized. The parameters associated with the “randomization” of the test modules, the inquiries therein, or both, will be described later in more detail.

In accordance with the present invention, the computer system is programmed and configured to calculate an individual's Current Brain Profile by a mathematical manipulation of the scores from its associated test session's test modules—hereinafter referred to as the “Current Test Module Scores”. The mathematical manipulation of theses scores can be accomplished in a number of different ways. After reading this description, numerous computation methods that can be employed for making this calculation will become apparent to those skilled in the art. All such methods, be they past, present or future, are deemed to be encompassed by the present invention.

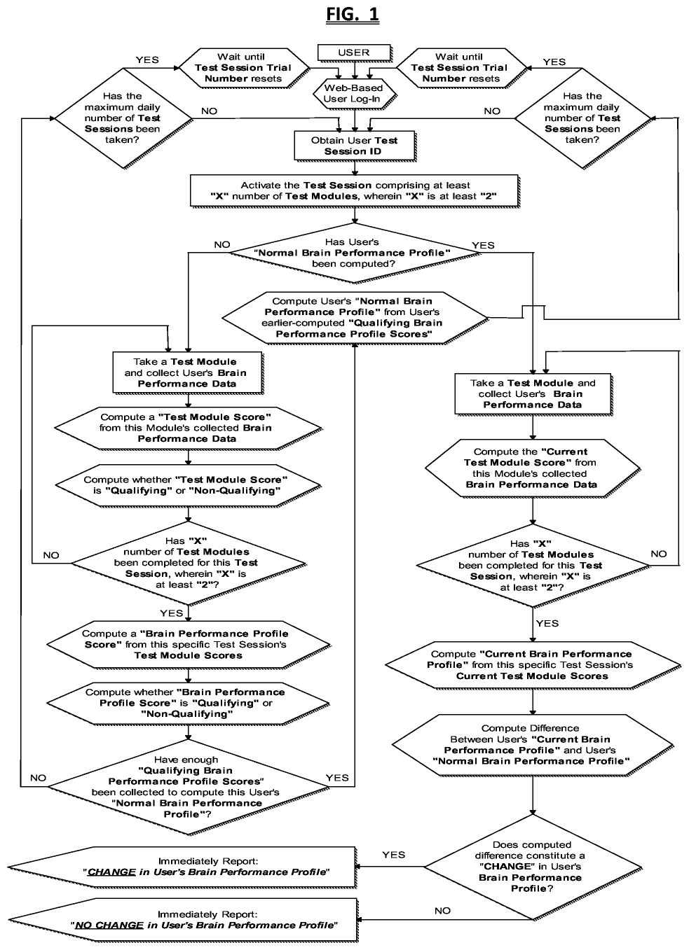

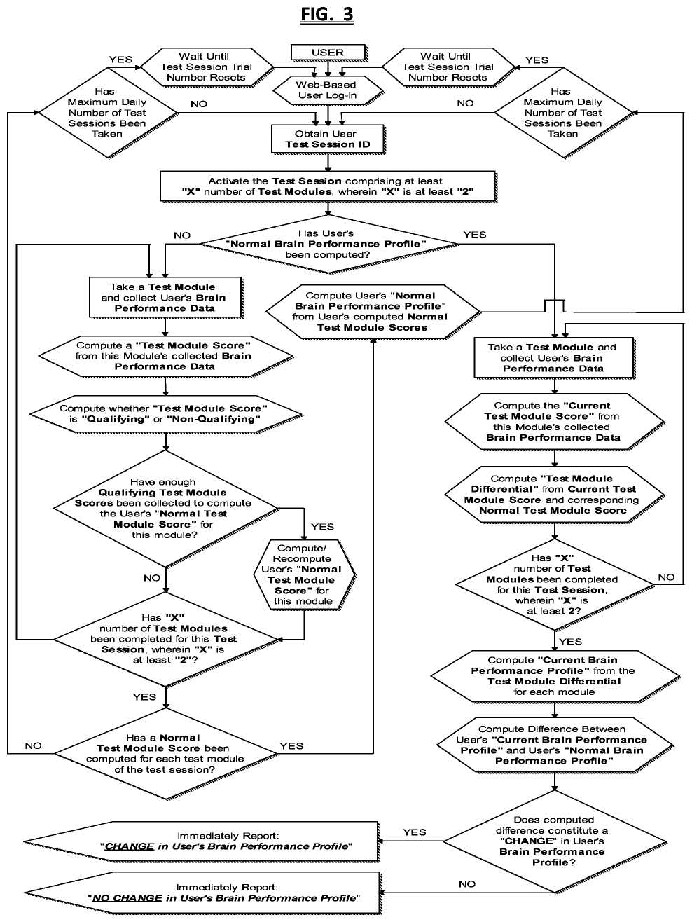

That being said, one particular method of computing an individual's Current Brain Profile comprises the computer system implementation of the same formulas as employed when calculating the individual's “Qualifying Brain Profiles” and/or “Non-Qualifying Brain Profiles”—see, e.g., FIG. 1. Moreover another particular method of computing an individual's Current Brain Profile comprises the computer system first calculating a “Test Module Differential Score” (defined infra) for each module by a mathematical manipulation of the module's Current Test Module Score and its corresponding “Normal Test Module Score” (defined infra). Then, once a Test Module Differential Score has been calculated for each module, the computer system is programmed and configured to compute the individual's Current Brain Profile from a mathematical manipulation thereof—see, e.g., FIG. 3. After reading this description, numerous computation methods that can be employed for making these calculations will become apparent to those skilled in the art. All such methods, be they past, present or future, are deemed to be encompassed by the present invention.

Regardless of how an individual's Current Brain Profile is calculated, as the number of times the present invention is employed to compute the same increases, so does the ability to detect subtle changes in an individual's Brain Profile. Accordingly, while a certain level of accuracy in detecting Brain Profile changes can be achieved by merely having the computer system calculate an individual's Current Brain Profile on an annual basis, in order to increase to probability of detecting a possible malady at a relatively early stage, one's Current Brain Profile should be calculated on at least a semi-annual basis; preferably, on at least a quarterly basis; more preferably, on at least a monthly basis; and even more preferably, on at least a weekly basis.

In circumstances, where there is perceived to be a problem with the brain, or where an individual is or has been exposed to an environment which can result in a brain function-altering injury (e.g., environments where an individual is subjected to bumps, blows, or jolts to the head), it is further preferred to have one's Current Brain Profile calculated on a daily basis. Moreover, since the tests encompassed by the present invention are non-invasive and relatively quick and simple to take, it is even possible for one's Current Brain Profile to be calculated multiple times during the same day, if necessary or desired.

The calculation of one's Current Brain Profile can also be performed at certain prescribed times. For example, for those in the transportation industries (e.g., aircraft/spacecraft crew, air traffic controllers, watercraft operators, truck drivers, train engineers, mass transit drivers, taxi drivers, etc.), since the results of the tests encompassed by the present invention are relatively instantaneous and provide a cumulative, objective and detailed characterization of the taker's unique Brain Profile at that particular point in time, the tests can easily be administered or taken such that one's Current Brain Profile is calculated immediately before beginning a shift or excursion to determine whether the individual is fit to undertake the task at hand.

In the preceding example, an individual could be requested to take a test encompassed by the present invention prior to undertaking a dangerous and demanding task to see if their Brain Profile has changed. However, since the test merely compares one's Current Brain Profile to his or her earlier-computed Normal Brain Profile, the tests of the present invention can also identify whether that individual's Current Brain Profile is better than their Normal one. Depending upon the brain functions being tested and monitored by a particular test session encompassed by the present invention, this can be an extremely useful tool for a number of reasons. For example, since the tests of the present invention can be configured to test and monitor an individual's brain function pertaining to their hand-eye coordination, this can assist a coach (e.g., a football coach, a soccer coach, a hockey coach, a lacrosse coach, a volleyball coach, a baseball coach, a basketball coach, a wrestling coach, etc.) to determine which particular athlete may perform better than another athlete in that day's event. Specifically, if a hockey coach has two equally talented goalies, the tests of the present invention can provide the coach with immediate and objective information as to which particular goalie's hand-eye coordination seems to be better on that particular day.

In addition to the foregoing, there can be other important reasons for having an athlete take a test encompassed by the present invention before and/or after he or she participates in a sporting event or activity. For example, if a 10-year old child is preparing to participate in some type of a sports-related event (e.g., cheerleading, football, soccer, hockey, lacrosse, volleyball, baseball, basketball, wrestling, etc.), the parent can easily have their child take a test encompassed by the present invention immediately before the event, and again upon his/her return, to see whether their Brain Profile has changed. If a change is detected, the parent now has the ability to immediately question the child to see if the change can be due to some sort of a brain trauma; and if so, to immediately seek medical attention therefor.

It is important to note that the present invention is not intended to be a medical diagnosis of a brain injury, problem, or ailment. Rather, it is merely a means of monitoring an individual's Brain Profile, so as to determine whether, and to what extent, there are changes to their brain functions making up the same. Once a change is detected, it is up to the individual, their parent, their guardian, their caregiver, or some other third party monitoring the results, to determine the cause for the change and/or to seek the appropriate medical attention. Medical attention can include calling an ambulance, taking the potentially injured party to an Emergency Room, or taking the potentially injured party to a concussion clinic, such as an ImPACT Concussion Clinic. The choice of medical attention employed, if any, is up to the discretion of the individual, their parent, their guardian, their caregiver, or some other third party monitoring the test results.

Simply put, the present invention can be described as functioning like a “Thermometer for the brain”℠, with the taker's Normal Brain Profile being their own uniquely-specific “98.6° F.”. In other words, just as a conventional thermometer, the methodologies encompassed by the present invention identify whether the takers' Brain Profile is off the particular norm that they created for themselves. Also like a conventional thermometer, the methodologies encompassed by the present invention are non-invasive, quick and simple to use, do not need to be administered or evaluated by a trained professional, and provide consistent and relatively instantaneous results.

However, unlike a conventional thermometer, the Normal Brain Profiles established by the methodologies of the present invention are specific and unique to each individual taker. As such, even if an individual's Normal Brain Profile may seem to be fairly good, or poor—when compared to the Normal Brain Profiles of others—the present invention's tests focus on how the individual's Current Brain Profile compares to their own specific Normal Brain Profile. In other words, when practicing the present invention, those taking the tests encompassed thereby are serving as their own control. That way, even minor changes to one's Brain Profile can be detected by the methodologies of the present invention. This, in turn, minimizes the instances of “false negatives” (i.e., an indication that there is no change detected to one's Brain Profile) or “false positives” (i.e., an indication that a change has been detected to one's Brain Profile).

While there are definite advantages associated with having the individual establish their own Normal Brain Profile (i.e., their own unique “98.6° F.”), in certain embodiments of the present invention, additional calculations can be made which result in an individual's Normal Brain Profile being modified as that individual progresses through their life. In other words, in these embodiments, the calculations made to establish an individual's Normal Brain Profile, are also used to make ongoing modifications thereto.

This particular embodiment of the invention further enhances the ability to detect minor changes to one's Brain Profile, regardless of their age. For example, it is expected that the Normal Brain Profile of a 10-year old person will be different from that, when the person turns 20, 40, or 60 years old. It is also possible for an individual's Normal Brain Profile to vary within a relatively short period of time (i.e., over a period of days, weeks, or months). As such, by employing the methodologies of these particular embodiments of the present invention, periodic adjustments are continuously made to that individual's Normal Brain Profile. This, in turn, enables the present invention to detect minor changes thereto, throughout the individual's life; and, as above, further minimizes the instances of “false negatives” or “false positives”.

With all that being said, in other embodiments of this invention, the Normal Brain Profile of an individual taker can also be compared to those of a set population of other takers. Such embodiments can be an extremely useful tool in circumstances where an individual who is taking the test for the first time, already has an unusually “impaired” or “enhanced” Brain Profile. Specifically, while the Brain Profile of a particular individual may appear to be unimpaired or unenhanced since his or her scores are relatively constant over a period of time, by employing these particular embodiments, the present invention can detect that the individual's Brain Profile may actually be one which is a result of an ailment, earlier injury to the brain, or some other brain-affecting malady.

### Methodologies of the Present Invention:

As mentioned above, the present invention pertains to, among other things, computer-based methods, systems, and mediums for identifying a change to the Brain Profile of an individual, wherein the change is determined by employing a computer system to perform at least the following tasks: a change to an individual's Brain Profile, wherein the change is determined by employing a “computer system” (defined infra.) to perform at least the following tasks: to establish an individual's Normal Brain Profile through the implementation of a test session taken at least two separate times; to establish the individual's Current Brain Profile through the implementation the test session taken at a time after their Normal Brain Profile has been established; and, to calculate whether there is a change to the individual's Brain Profile through a mathematical manipulation of their Normal Brain Profile and their Current Brain Profile, wherein any detected change can be the result of an impairment or enhancement to at least one of the individual's neuromotor or cognitive brain functions being tested.

Specifically, the computer-based methods, systems and medium encompassed by the present invention comprise providing means for the computer system to establish an individual's Normal Brain Profile and their Current Brain Profile through the implementation of a test session taken at least three separate times. Illustrative, but non-limiting, examples of such means will be described later in more detail.

In order to establish the individual's Normal and Current Brain Profiles the computer-based methods, systems and medium encompassed by the present invention further comprise providing means for an individual to initiate and take the test session at least three separate times on the computer system. Illustrative, but non-limiting, examples of such means will be described later in more detail.

The test session employed when practicing the present invention comprises at least a first brain function test module and a second brain function test module. The first and second test modules differ from one another, and are each designed to collect certain brain performance data from an individual, wherein the collected brain performance data correlates to at least one of the individual's neuromotor or cognitive functions.

As the various test sessions are being taken by the individual, the computer-based methods, systems and medium encompassed by the present invention also comprise providing means for the computer system to collect the individual's brain performance data, and to compute the individual's first and second test module scores through a mathematical manipulation of the collected data. Illustrative, but non-limiting, examples of such means will be described later in more detail.

If the computer system is in the process of establishing the individual's Normal Brain Profile, once a test module score has been computed for each of the test session's modules, the computer-based methods, systems and medium encompassed by the present invention further comprise providing means for the computer system to compute a Brain Profile Score, a Normal Test Module Score (defined infra), or both through a mathematical manipulation of earlier-computer test module scores. If the computer system computes a Brain Profile Score, the computer-based methods, systems and medium encompassed by the present invention also comprise providing means for the computer system to compute the same through a mathematical manipulation of all of that test session's test module scores. If, however, the computer system computes Normal Test Module Scores, the computer-based methods, systems and medium encompassed by the present invention further comprise providing means for the computer system to compute the same through a mathematical manipulation of at least two of each module's test module scores computed from test sessions completed at different times. Illustrative, but non-limiting, examples of all such means will be described later in more detail.

If the computer system is in the process of establishing the individual's Current Brain Profile, once a test module score has been computed for each of the test session's modules (hereinafter referred to as a module's “Current Test Module Score”), the computer-based methods, systems and medium encompassed by the present invention further comprise providing means for the computer system to compute the individual's Current Brain Profile, Test Module Differentials (defined infra), or both. Illustrative, but non-limiting, examples of such means will be described later in more detail.

If the computer system computes a Test Module Differential for each of that test session's modules, the computer-based methods, systems and medium encompassed by the present invention also comprise providing means for the computer system to compute the same through a mathematical manipulation of each module's Current Test Module Score and its corresponding, earlier-computed, Normal Test Module Score. Illustrative, but non-limiting, examples of such means will be described later in more detail.

In accordance with the present invention, an individual's Current Brain Profile is computed from a test session's Current Test Module Scores, its Test Module Differentials, or both. If the computer system computes an individual's Current Brain Profile Score from Current Test Module Scores, the computer-based methods, systems and medium encompassed by the present invention also comprise providing means for the computer system to compute the same through a mathematical manipulation of all of that test session's Current Test Module Scores. If, however, the computer system computes an individual's Current Brain Profile Score from Test Module Differentials, the computer-based methods, systems and medium encompassed by the present invention further comprise providing means for the computer system to compute the same through a mathematical manipulation of all of that test session's Test Module Differentials. Illustrative, but non-limiting, examples of such means will be described later in more detail.

Regardless of how they are calculated, once the individual's Normal and Current Brain Profile Scores are established, the computer-based methods, systems and medium encompassed by the present invention further comprise providing means for the computer system to compute the difference between these two scores, and to compute whether this difference constitutes a change in the individual's Brain Profile. Illustrative, but non-limiting, examples of all such means will be described later in more detail.

Once the computer system computes whether or not a change has occurred in the individual's Brain Profile, the computer-based methods, systems and medium encompassed by the present invention also comprise providing means for the computer system to compute the difference between these two scores, and to report the same. Illustrative, but non-limiting, examples of all such means will be described later in more detail.

The computer system employed when practicing this invention comprises a personal computing device and a server device. Examples of personal computing devices and server devices that can be used when practicing the present invention are described later, in more detail.

In the computer system of the present invention, the personal computing device and the server device are both configured and programmed such that they are able to perform at least the following tasks: to process a user's input responses, and to receive and send data and information to and from one another via a communications network. Examples of communications networks that can be used when practicing the present invention are described later, in more detail.

When practicing certain embodiments of this invention, the computer system's personal computing device is first connected to the system's server device, via a communications network. After the connection is made, certain user-specific data and information is exchanged between the personal computing device and the server device.

In order to activate and identify a particular test session encompassed by the present invention, the users are prompted to input certain personal data. Once activated and identified, the various modules of the particular test session presents to the user specific inquiries which are directly related to the performance levels of at least one of their brain functions. Next, through a series of programmed mathematical manipulations, the computer system collects the user's brain performance data and computes therefrom a cumulative score for that particular test module (i.e., a “Test Module Score”). As will be seen later in the description of this invention, a Test Module Score can be classified as a “Qualifying Test Module Score”, a “Non-Qualifying Test Module Score”, a “Normal Test Module Score” or a “Current Test Module Score”. After reading this description, numerous computation methods for calculating all of these scores will become apparent to those skilled in the art. All such methods, be they past, present or future, are deemed to be encompassed by the present invention.

When a Test Module Score is computed as part of the process associated with the establishments of an individual's Normal Brain Profile, after the Test Module Score is calculated, the computer system is programmed and configured to determine whether it is a “Qualifying Test Module Score” or a “Non-Qualifying Test Module Score”. If all of the Test Module Scores of a particular test session are Qualifying Test Module Scores, the computer system is programmed and configured to compute a “Qualifying Brain Profile Score” for that session. However, if at least one of the Test Module Scores of a particular test session is a Non-Qualifying Test Module Score, then the computer system is programmed and configured to compute a “Non-Qualifying Brain Profile” for that session.

Once at least two earlier-computed Qualifying Brain Profiles have been collected for an individual, the computer system can be programmed and configured to compute the user's Normal Brain Profile therefrom. After the user's Normal Brain Profile has been computed, the Brain Profiles computed from all subsequently-completed test sessions become the user's Current Brain Profiles. Through a mathematical manipulation of the user's Normal Brain Profile, and any subsequently-computed Current Brain Profile, the computer system can monitor and report when, and to what extent, their Brain Profile has changed. Regarding all of the aforementioned computation methods, after reading this description, numerous methods that can be employed when making these calculations will become apparent to those skilled in the art. All such methods, be they past, present or future, are deemed to be encompassed by the present invention.

The test sessions encompassed by the present invention can be initiated a number of different ways. For example, in certain embodiments, the test session is initiated by the user entering information on the system's personal computing device so as to communicate with the system's server device via a communications network. In response to that communication, the system's server device can provide the user with an “identifier” that is specific to that user, and to that user's particular test session being activated. This test session identifier is then communicated back to the system's personal computing device via a communications network. Once the system's personal computing device receives the specific test session identifier, the user is able to activate the test session.

The activated test sessions comprise at least two separate and distinct brain function test modules, each of which is designed to collect certain brain performance data from an individual user. The brain performance data collected from both of the at least two test modules correlate to at least one of the user's neuromotor or cognitive brain function.

While the at least two test modules making up a particular test session are separate and distinct from one another, it is, none-the-less, within the scope of this invention for at least some of the neuromotor and/or cognitive function(s) being tested in one test module to be the same exact one(s) being tested in the other test module(s). In such an embodiment, the only requirement is that the manner in which the function(s) is/are tested in the at least two test modules are different from one another.

That being said, as the number of brain functions being tested in a particular test session increases, so does the test's ability to detect more potential injuries, diseases, disorders, and maladies of the brain. Accordingly, in certain preferred embodiments of this invention, at least a portion of the neuromotor and/or cognitive functions tested in one of the at least two test modules is different from those tested in at least one of the other(s). As such, in these preferred embodiments, at least two separate neuromotor and/or cognitive functions are being tested in one particular test session.

It is, however, within the scope of these particular embodiments for the at least two test modules to each test multiple neuromotor and/or cognitive functions. It is also within the scope of these particular embodiments for the functions tested in the one of the at least two test modules to be the same exact ones tested in the other test module(s). Under such circumstances, and as stated above, the only requirement is that the manners in which the functions are tested in the various modules must differ from one another.

In certain preferred embodiments, the grouping of test modules employed within a particular test session are such that at least two; preferably, at least four; and more preferably, at least six separate neuromotor and/or cognitive functions are tested. Examples of the types of brain functions that can be monitored and tested in accordance with practicing the present invention include, without limitation, the following: visual learning, short-term memory recall, long-term memory recall, image recognition, anticipation timing, executive functioning, visual perception, spatial visual working memory, eye-hand coordination, manual dexterity, information processing, vigilance, numerical working memory, digit span forwards and backwards, neuromotor channel capacity, coordination, confidence, number and figure comparison, synonym reasoning, and balance.

A human's neuromotor and cognitive brain functions, including those set out above, are generally organized as falling within one of many separate and distinct domains. These domains, and a sampling of some of the functions associated therewith, are as follows: “Executive” (e.g., verbal fluency, confidence, and make competent decisions and judgments), “Processing Speed” (e.g., number and figure processing and comparison); “Episodic Memory” (e.g., episode recall); “Echoic Memory” (e.g., sound recall); “Visual-Spatial” (e.g., mental rotations and ability to visualize, manipulate, and recreate objects); “Working Memory” (e.g., digit span forwards and backwards, recall previous experiences, and learn new information); “Semantic Memory” (e.g., synonym reasoning); “Attention” (e.g., concentrate and focus on current information); “Verbal Fluency” (e.g., use of language quickly and accurately); “Orientation” (e.g., awareness of time, place, situation and self); and “Cerebral” (e.g., motor functions such as balance).

Since the test modules of the present invention target at least one brain function, practicing this invention can, not only identify whether and to what extent there is a change to the user's Brain Profile; but also, assist in identifying what part of the user's brain may be associated with that change. A medical professional can, in turn, use this information to map out an examination and treatment regimen.

The number and configuration of the test modules employed when practicing this invention is a function of the number of, and/or particular, brain functions that one wishes to test and monitor. Therefore, while the present invention requires the implementation of at least two separate and distinct brain function test modules, the invention is, in no way, limited to that number.

As mentioned above, the test session being employed when practicing this invention must include at least 2 separate and distinct test modules. However, in those instances where it is desirable to establish and monitor a more comprehensive Brain Profile, the test sessions being employed should include at least 4 separate and distinct test modules cumulatively testing the brain functions of at least 2 domains of the brain; more preferably, at least 6 separate and distinct test modules cumulatively testing the brain functions of at least 3 domains of the brain; and even more preferably, at least 8 separate and distinct test modules cumulatively testing the brain functions of at least 4 domains of the brain. One preferred example of a test session, which includes a particular grouping of the test modules designed to establish, test, and monitor a relatively comprehensive Brain Profile, will be described later in more detail.

Test sessions taken in accordance with this invention can be ones which are located either on the system's server device, on the system's personal computing device, or on both. If the test session is located on the system's personal computing device, it can be placed thereon in a number of different ways. For example, in such an embodiment, the test session can be one which was earlier downloaded onto the computer system's personal computing device, either from the system's server device, or from some other sort of a storage medium (e.g., a CD, a DVD, a flash drive, an application or applet—a.k.a. an “app”, etc.). A test session can also be one which is downloaded to the system's personal computing device, from the system's server device, each time that a new test session is to be taken.

It is within the scope of this invention for the at least two test modules making up a particular test session to be presented in the same order each time the test is taken. It is also within the scope of the present invention for the particular test modules, themselves, to be exactly the same each time the test is taken. That being said, is certain embodiments, the test sessions taken in accordance with the present invention are randomized. In such embodiments, the randomization can result by the order of the particular test modules being randomized, or by there being a randomization of the inquiries within the individual test modules themselves, or both.

If employed, randomization can occur a number of different ways. For example, in one embodiment, the first time a user accesses the system's server device, a “randomizable” test session can be downloaded onto their personal computing device. When used herein, the term “randomizable”, as it refers to a test session, pertains to a test session that can be changed with each new activation. This reduces the possibility of a user memorizing any particular test session.

The manners in which such randomization can be accomplished, and still fall within the scope of this invention, are limitless. For example, randomization can occur by the system's server device transmitting changes to the system's personal computing device each time a new test session is activated. Moreover, it can also occur by potential changes to subsequent test sessions being downloaded onto the system's personal computing device with the downloading of the initial test session, along with instructions as to how the randomization is to occur each time a new test session is activated. It can also occur by some sort of a combination of the former two examples.

After a specific test session has been activated, the testing, monitoring, and evaluation process can begin. However, prior to being able to identify any changes to one's Brain Profile, the present invention needs to first establish the individual's Normal Brain Profile. As mentioned above, when calculating the user's Normal Brain Profile, the computer system is programmed and configured to first compute a user-specific baseline from the scores of at least two of the user's earlier-computed Qualifying Brain Profiles. Thereafter, the computer system is then programmed and configured to compute what constitutes the consistent range of scores which are necessary to establish the user's Normal Brain Profile.

As such, the tests encompassed by the present invention can identify a change in a user's Brain Profile after only the third time the test is taken. In accordance with this particular embodiment, Qualifying Test Module Scores collected from the user's first completed test session are used by the computer system to compute their first Qualifying Brain Profile. Then, Qualifying Test Module Scores collected from the user's second completed test session are used by the computer system to compute their second Qualifying Brain Profile. The computer system is then programmed and configured to compute the user's Normal Brain Profile from a mathematical manipulation of their earlier-computed first and second Qualifying Brain Profiles. Once the user's Normal Brain Profile is computed, the test module scores from their third completed test session (i.e., the user's “Current Test Module Scores” defined supra) are employed by the computer system to compute the user's Current Brain Profile. The computer system is then programmed and configured to compute whether there is a change in the user's Brain Profile through a mathematical manipulation of their earlier-computed Normal Brain Profile and their recently-computed Current Brain Profile.

Regardless of the number of Qualifying Brain Profile scores which is used by the computer system to calculate the user's Normal Brain profile score, the computer system is programmed and configured to first compute a user-specific baseline therefrom. Thereafter, the computer system is programmed and configured to calculate the consistent range of scores making up the same.

It is within the scope of this invention for the computer system to calculate what constitutes the user's Normal Brain Profile “consistent range” of scores, after a certain number Qualifying Brain Profile Scores have been collected, or after the user's Qualifying Brain Profile Scores consistently fall within a particular range, or after some combination of both.

As the user becomes more familiar with a particular test session, it is expected that the “consistent range” of their Qualifying Brain profile Scores will begin to, not only improve, but also narrow. This will produce a more accurate Normal Brain Profile for that individual, which, in turn will provide a more accurate, means of identifying subtle changes in their Brain Profile.

An important distinction between the prior art testing methodologies and those encompassed by the present invention is that, when the prior art testing methodologies employ the concept of “baselines”, they are, and remain, exactly just that—a “line”. Thus, if the prior art testing methodologies have any level of sensitivity associated therewith, it will be highly improbable for an individual who is taking consecutive tests to have identical scores. As such, with the prior art testing methodologies, there typically needs to be some sort of a trained professional evaluating the test results to see if the subtle changes—which will almost always be present—are caused by something that requires medical attention.

However, unlike the prior art testing methodologies, with those encompassed by the present invention, a user's Normal Brain Profile is not a “baseline”; but rather, a “consistent range” of scores whose width is determined by the individual him or herself. As such a trained professional is not required to determine whether the individual is “off” their norm, even when subsequently-generated test scores are not identical to one another. Specifically, in accordance with the present invention, a change in an individual's Brain Profile is only reported when a particular Current Brain Profile for that individual falls outside of the earlier-computed “consistent range” of scores which constitutes their Normal Brain Profile, as opposed to merely being “off” a particular “baseline”—which will almost always be the case.

The consistent range of scores which make up a user's Normal Brain Profile can be a function of time/number sensitivity, score sensitivity, or both. For example, it is within the scope of the present invention for the computer system to calculate what is to be the consistent range of scores which make up an individual's Normal Brain Profile, by merely considering the number of times a Qualifying Brain Profile Score is calculated for that individual. As the number of Qualifying Brain Profile Scores used in this calculation increases, the narrower the consistent range of scores is expected to be. In such an embodiment, a consistent range of scores can be calculated from only 2 earlier-computed Qualifying Brain Profile Scores. However, preferably, at least 10 Qualifying Brain Profile Scores; more preferably, at least 20 Qualifying Brain Profile Scores; and even more preferably, at least 30 Qualifying Brain Profile Scores are employed in this calculation.

In order to minimize the possibility of test scores being skewed due to factors such as fatigue or complacency, when time is the determining factor for calculating an individual's Normal Brain Profile, the Qualifying Brain Profile Scores used in its calculation are generally collected from test sessions completed on separate days. The preferred span over which such test sessions are completed is dependent, in part, upon the number of Qualifying Brain Profile Scores used in the calculation of the user's Normal Brain profile. For example, if only 2 Qualifying Brain Profile Scores are used in this calculation, the test sessions from which those scores result should be completed over at least a 2-day period of time. However, if at least 10 Qualifying Brain Profile Scores are used in this calculation, the test sessions from which those scores result are preferably completed over at least a 5-day period of time. Moreover, if at least 20 Qualifying Brain Profile Scores are used in this calculation, the test sessions from which those scores result are preferably completed over at least a 10-day period of time; and, if at least 30 Qualifying Brain Profile Scores are used in this calculation, the test sessions from which those scores result are preferably completed over at least a 15-day period of time.

The consistent range of test session scores can also be a result of score sensitivity. For example, it is within the scope of the present invention for the computer system to calculate what constitutes the consistent range of scores making up a user's Normal Brain Profile by having the computer system monitor earlier-computed Qualifying Brain Profile scores and compute when a certain pre-established percentage of those scores consistently falls within a particular range. After reading this description, numerous computation methods that can be employed for making this calculation will become apparent to those skilled in the art. All such methods, be they past, present or future, are deemed to be encompassed by the present invention

In such an embodiment, the percentage of earlier-completed Qualifying Brain Profile scores which need to fall within a consistent range is largely dependent upon the needs and requirements of the end user, as well as the particular variance of scores from the individual taker. It is also dependent upon the outlying scores from the test session modules, and how, and/or whether, they are considered in the computation of what constitutes the consistent range. For example, assume that after 10 completed test sessions, 100% of the user's Qualifying Brain Profile scores fall within a range of ±2× from their earlier-computed baseline. However, then assume that, if the outlying scores (i.e., the single worst brain performance data scores from the various test modules making up the test session) are ignored, 100% of the user's remaining Qualifying Brain Profile scores fall within a range of ±1× from their baseline. In this example, if the computer system is be programmed and configured to consider 100% of all Qualifying Brain Profile scores, the Normal Brain Profile for that user will be within a range which is a factor of ±2× from their baseline. However, if the computer system is be programmed and configured to ignore the outlying scores and only consider 100% of the Qualifying Brain Profile scores which remain, the Normal Brain Profile for that user will be within a range which is a factor of ±1× from their baseline.

When practicing any embodiment of this invention, how, and/or whether, the computer system is programmed and configured to consider outlying scores, as well as what are the parameters of any such outlying score, is largely dependent upon the needs and requirements of the end user. That being said, all such programming and configuration is deemed to be encompassed by the present invention.

Although the number of times a test session is completed is not critical when employing the embodiment wherein the consistent range computation is based solely upon score sensitivity, in one preferred embodiment, an upper limit of testing times can be set to aid in determining whether the user's Brain Profile is already in an overly impaired or enhanced state; or whether the user is not correctly completing the test session's modules. In such a preferred embodiment, if after a pre-established number of times that the test is taken, a Qualifying Brain Profile cannot be computed, the computer system can be programmed and configured to report to the user that a problem has been detected; and that the problem can be either that the user is not taking the test sessions correctly, or that potential change to the user's Brain Profile may already exist.

The consistent range of test session scores can also be a result of both, time/number sensitivity and score sensitivity. Here, the computer system can be programmed and configured to collect a pre-established number of earlier-computed Qualifying Brain Profiles. Then, after that number has been collected, the computer system can be programmed and configured to determine whether the Qualifying Test Module Scores making up the individual collected Qualifying Brain Profiles fall within a pre-established range. If so, the computer system can be programmed and configured to use the collected Qualifying Brain Profiles to compute the user's Normal Brain Profile. However, if not, the computer system can be programmed to set an additional pre-established number of test sessions that the user needs to be completed, before this consistent range-evaluation is repeated. This process can continue until a consistent range of score is established.

Once computed, an individual's Normal Brain Profile can equate to a static range of scores, or to a range of scores that periodically changes over the user's lifetime. In instances where it is desirable to detect very subtle changes in one's Brain Profile, it is preferred that their Normal Brain Profile be recalculated at set intervals to determine whether there is an upward or downward trend in the same. For example, the computer system of the present invention can be programmed and configured collect a certain set of previously-completed Current Brain Profile scores; compute a cumulative score from that particular grouping; compare that computed cumulative score to the user's most recently-computed Normal Brain Profile score; and compute a new Normal Brain Profile score for that user which takes into consideration the recently-computed cumulative score from the aforementioned grouping of Current Brain Profile scores. Such a periodic re-computation enables the present invention to more accurately determine whether a subsequently-computed Current Brain Profile score falls within or outside of the user's most current Normal Brain Profile. After reading this description, numerous computation methods that can be employed for making this calculation will become apparent to those skilled in the art. All such methods, be they past, present or future, are deemed to be encompassed by the present invention.

In embodiments where the computer system recalculates an individual's Normal Brain Profile, the interval over which the grouping of earlier-computed Current Brain Profile scores used in this calculation are collected can be a factor of time, the number of scores collected, or both. If a “time” factor is employed, the interval from which the grouping of earlier-completed Current Brain Profiles is collected for this re-calculation of the individual's Normal Brain Profile should be from between 1 week and 1 year; preferably, from between 2 weeks and 9 months; more preferably, from between 3 weeks and 6 months; and even more preferably, from between 1 month and 3 months. If a “number” factor is employed, the interval from which the grouping of earlier-completed Current Brain Profiles is collected for this re-calculation of the individual's Normal Brain Profile should be from between 7 and 360; preferably, from between 14 and 270; more preferably, from between 21 and 180; and even more preferably, from between 30 and 90. If both a “time” and “number” factor are employed, the computer system can be programmed and configured to determine whether, after a certain period of time has elapsed, a pre-determined number of earlier-computed Current Brain Profile scores has been collected. If so, the computed system can be programmed and configured to re-calculate the individual's Normal Brain Profile at that point in time. However, if enough earlier-computed Current Brain Profile scores have not yet been collected, the computer system can be programmed and configured to set an additional time period over which to collect the same. This process can continue until enough data exists to re-calculate the individual's Normal Brain Profile.

Since a user's initial Normal Brain Profile score is initially calculated from earlier-computed Qualifying Brain Profiles, and since each such earlier-computed Qualifying Brain Profile results from Qualifying Test Module Scores, the computer system of the present invention can also be programmed and configured to calculate a “normal” score for each of the test modules making up a particular test session, hereinafter referred to as a user's “Normal Test Module Score”.

In accordance with the present invention, a Normal Test Module Score is calculated from a mathematical manipulation of at least two earlier-computed Qualifying Test Module Scores for a particular module. As the number of Qualifying Test Module Scores used in the calculation of a module's Normal Test Module Score increases, so does the accuracy of the results and the sensitivity of the test. Accordingly, although a module's Normal Test Module Score can be calculated from only 2 earlier-computed Qualifying Test Module Scores, preferably, at least 10 Qualifying Test Module Scores; more preferably, at least 20 Qualifying Test Module Scores; and even more preferably, at least 30 Qualifying Test Module Scores are employed in this calculation. After reading this description, numerous computation methods that can be employed for making this calculation will become apparent to those skilled in the art. All such methods, be they past, present or future, are deemed to be encompassed by the present invention

In order to minimize the possibility of test scores being skewed due to factors such as fatigue or complacency, the Qualifying Test Module Scores used in the calculation of their respective Normal Test Module Scores are generally collected from test sessions completed on separate days. The preferred span over which such test sessions are completed is dependent, in part, upon the number of Qualifying Test Module Scores used in the calculation of each module's Normal Test Module score. For example, if only 2 Qualifying Test Module Scores are used in this calculation, the test sessions from which those scores result should be completed over at least a 2-day period of time. However, if at least 10 Qualifying Test Module Scores are used in this calculation, the test sessions from which those scores result are preferably completed over at least a 5-day period of time. Moreover, if at least 20 Qualifying Test Module Scores are used in this calculation, the test sessions from which those scores result are preferably completed over at least a 10-day period of time; and, if at least 30 Qualifying Test Module Scores are used in this calculation, the test sessions from which those scores result are preferably completed over at least a 15-day period of time.

Since a test session comprises at least two test modules, it is possible that, when practicing this embodiment, each module's Normal Test Module Score will not be calculated at the same time (i.e., each time a test session is completed, it is possible that not all of the Test Module Scores for that session will be Qualifying Test Module Scores). Under such a circumstance, the computer system can be programmed and configured to either store a module's particular Normal test Module Scores until one is obtained for each of the session's modules. However, in a preferred embodiment, until such time that the final module's Normal Test Module score is calculated, the computer system can be programmed and configured to re-calculate the earlier-computed Normal Test Module Scores of the other modules.

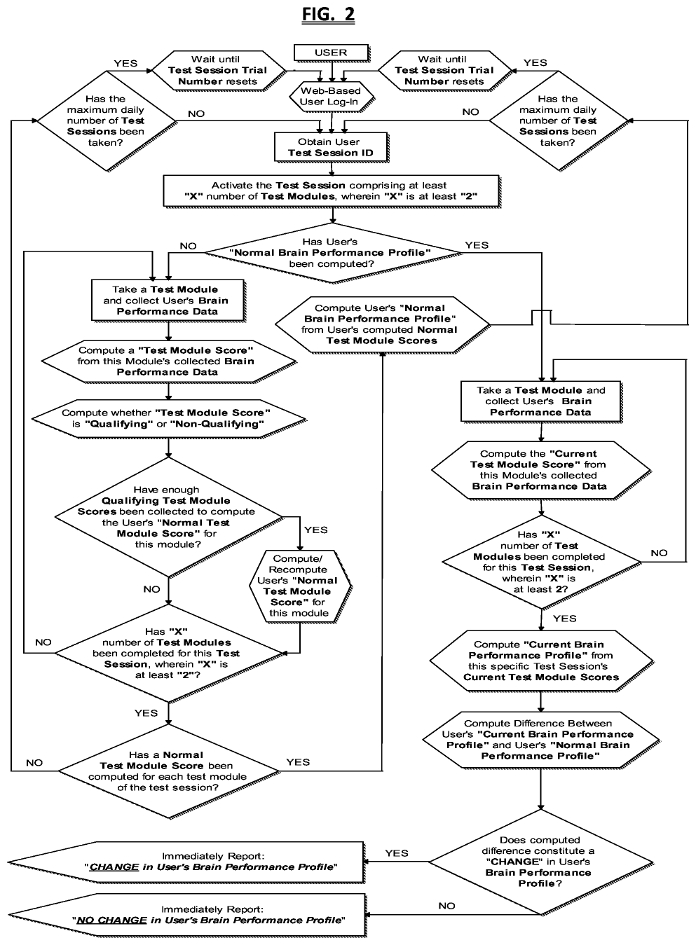

Once the test session's final Normal Test Module Score has been calculated, the computer system can be programmed and configured to calculate the user's Normal Brain Profile therefrom (see, e.g., FIG. 2). In addition to being used to calculate a user's Normal Brain Profile, the Normal Test Module Scores for each module can also be employed by the computer system to monitor how an individual's brain functions are performing by comparing subsequently-calculated Current Test Module Scores to that same module's earlier-calculated Normal Test Module Score. Moreover, Normal Test Module Scores can even be employed in the computation of the user's Current Brain Profile by computing each module's Test Module Differential for a comparison of its Normal Test Module Score and its Current Test Module Score (see, e.g., FIG. 3). After reading this description, numerous computation methods that can be employed for making the aforementioned calculations will become apparent to those skilled in the art. All such methods, be they past, present or future, are deemed to be encompassed by the present invention.

Regardless of how an individual's Normal Brain Profile score has been calculated, once established, the same test session used in its calculation should be employed to calculate the user's Current Brain Profile. Note, while the phrase “the same test session” encompasses subsequent test sessions wherein neither the order of the test modules, nor the inquiries therein, are randomized, it is not limited to such a definition. Specifically, the phrase “the same test session” also encompasses subsequent test sessions wherein: (a) only the order of the test modules is randomized; (b) only the inquiries within the particular test modules are randomized; or (c) both, the order of the test modules and the inquiries therein, are randomized.

The present invention then provides means for calculating whether there is a change to the user's Brain Profile through a mathematical manipulation of the user's earlier-computed Normal Brain Profile, and their recently-calculated Current Brain Profile. After reading this description, numerous computation methods that can be employed for making this calculation will become apparent to those skilled in the art. All such methods, be they past, present or future, are deemed to be encompassed by the present invention.

As mentioned above, while at least two test modules need to be employed when practicing this invention, the actual number of modules making up a particular test session is a function of the number of, and/or particular, brain functions that one wishes to test and/or monitor. For example, if an end user wishes to utilize the present invention to only monitor those brain functions that might have an effect on a user working in the transportation industry (e.g., aircraft/spacecraft crew, air traffic controllers, watercraft operators, truck drivers, train engineers, mass transit drivers, etc.), the test modules making up that particular test session can be selected and configured to collect brain performance data pertaining to those brain functions affected by stress, fatigue, sleep deprivation, alcohol consumption, and/or substance abuse.

In most instances where a comprehensive analysis of one's brain functions is desired, the number of modules employed in a particular test session will generally be more than two. FIG. 1 is a flow diagram illustrating a method and system of one particular embodiment of the present invention. Upon reading the description of FIG. 1, numerous other suitable methods and systems, which can be employed to determine whether there is a change to a user's Brain Profile, will become apparent to those skilled in the art. Such suitable methods and systems are deemed to be encompassed by the present invention.

In the system illustrated in FIG. 1, the user begins by employing the computer system's personal computing device to connect to the system's server device. This connection occurs via a communications network. Examples of personal computing devices, server devices and communication networks that can be used when practicing this invention are described later in more detail. However, for the purposes of continuing with the description of this example, the specific computing device, server device and/or communication network being employed are not critical.

Once the system's personal computing device and server device are connected to one another, the computer system prompts the user to employ a “Web-Based User Log-In”. The computer system is programmed and configured to confirm the user's identity from the data inputted in the Web-Based User Log-in. Once the user's identity is confirmed, the computer system is programmed and configured to set and provide the user with a uniquely-specific identifier for that particular test session (i.e., a “Test Session ID”). After the Test Session ID has been obtained by the user, the Test Session can be activated.

In the embodiment illustrated in FIG. 1, the activated Test Session comprises “X” number of Test Modules, wherein “X” is at least 2. As mentioned earlier, the present invention also encompasses embodiments wherein the test session is comprised of more than two test modules. In such embodiments, the steps set out below can be duplicated for each additional module being employed.

In the FIG. 1 embodiment, prior to initiating an activated test session, the computer system is programmed and configured to determine whether the user's Normal Brain Profile has already been established. For the purpose of continuing with the description of this example, it will be presumed that the answer to that determination is “NO”. Under such a circumstance, the computer system is programmed and configured to proceed with establishing the user's Normal Brain Profile.

In this example, the computer system computes the user's Normal Brain Profile by having the user complete “X” number of Test Sessions, wherein “X” is at least 2. Each of the Test Session's Test Modules are designed and configured to collect certain brain performance data from the user. The brain performance data being collected from these Test Modules correlates to at least one of the user's cognitive and/or neuromotor brain functions. These Test Modules are also separate and distinct from one another. Therefore, at least a portion of the brain performance data collected from one Test Module will differ from the brain performance data collected from the other(s).

As the user is taking the first Test Module, the computer system is programmed and configured to collect their responsive brain performance data. Once the user has completed the first Test Module, the computer system is programmed and configured to compute a Test Module Score for that particular module. This Test Module Score is computed from a mathematical manipulation of the particular module's collected brain performance data. After reading this description, numerous computation methods that can be employed for making this calculation will become apparent to those skilled in the art. However, for the purposes of continuing with the description of this example, the particular method used to compute the user's Test Module Scores is not critical.

Once a particular Test Module Score has been computed, the computer system is programmed and configured to determine whether the Test Module Score is a Qualifying Test Module Score or a Non-Qualifying Test Module Score (i.e., a Qualifying Test Module Score is one that can be used in the calculation of a Qualifying Brain Module Score or a Normal Test Module Score). After reading this description, numerous computation methods that can be employed to make such a determination will become apparent to those skilled in the art. However, for the purposes of continuing with the description of this example, the particular method used to classify a Test Module Score as Qualifying or Non-Qualifying is not critical.

After the Test Module Score has been designated as Qualifying or Non-Qualifying, the computer system is programmed and configured to determine whether all of the Test Modules making up the test session have been completed by the user. Since this invention requires that each Test Session comprises at least two Test Modules, and since the Test Module which was just completed was the first Test Module, at this point in the process, the answer to the question as to whether all of the Test Modules have been completed by the user will be “NO”. Accordingly, the computer system is programmed and configured to prompt the user to take and complete the remaining Test Modules making up the particular Test Session. As each Test Module is completed, the computer system is programmed and configured to compute that module's Test Module Score, and to designate that Test Module Score as Qualifying or Non-Qualifying.

Once all of the Test Modules for the particular Test Session have been completed, the computer system is programmed and configured to compute the user's Brain Profile Score for that particular Test Session. This Brain Profile Score is computed from a mathematical manipulation of the Test Session's earlier-computed Test Module Scores, regardless of whether they are Qualifying or Non-Qualifying. After reading this description, numerous computation methods that can be employed to make this calculation will become apparent to those skilled in the art. However, for the purposes of continuing with the description of this example, the particular method used to compute the user's Brain Profile Score is not critical.

After the Test Session's Brain Profile score has been computed, the computer system is programmed and configured to determine whether the Brain Profile Score is a Qualifying Brain Profile Score or a Non-Qualifying Brain Profile Score (i.e., a Qualifying Brain Profile Score is one that can be used in the calculation of the user's Normal Brain Profile). After reading this description, numerous computation methods that can be employed to make such a determination will become apparent to those skilled in the art. However, for the purposes of continuing with the description of this example, the particular method used to classify a Brain Profile Score as Qualifying or Non-Qualifying is not critical.

After the Brain Profile Score has been designated as Qualifying or Non-Qualifying, the computer system is programmed and configured to determine whether enough Qualifying Brain Profile Scores have been collected to compute the user's Normal Brain Profile. In accordance with the present invention, Since this invention requires that a user's Normal Brain Profile be calculated from at least two of the user's earlier-computed Qualifying Brain Profile Scores, and since the Brain Profile Score which was just computed was the user's first Brain Profile Score, even if it is a Qualifying score, the answer to the question as to whether enough Qualifying Brain Profile Scores have been collected to compute the user's Normal Brain Profile will be “NO”. Accordingly, the computer system is programmed and configured to prompt the user to take and complete at least one additional Test Session. As each Test Session is completed, the computer system is programmed and configured to compute that session's Brain Profile Score, and to designate that Brain Profile Score as Qualifying or Non-Qualifying.

In order to provide more accurate results, in certain preferred embodiments, the computer system is programmed and configured such that multiple Test Sessions are to be completed over multiple days. In these particular embodiments, as in the FIG. 1 example, the number of times a user can take a Test Session per day is limited. As such, if the user is prompted to take an additional Test Session, and if the maximum number of daily Test Sessions has not yet been reached, he or she can do so by merely obtaining a new, uniquely-specific Test Session ID. However, if the maximum number of daily Test Sessions has been reached, then the user will be prompted to wait for the Test Session Trial Number to reset. As such, the next time the user wishes take a new Test Session, he or she will need to access the computer system's server device with the system's personal computing device, and employ the Web-Based User Log-In to obtain a new Test Session ID.

Once enough of the user's earlier-computed Qualified Brain Profile scores have been collected (i.e., the answer to the question as to whether enough Qualified Brain Profile Scores have been collected to compute the user's Normal Brain Profile becomes “YES)”, the computer system is programmed and configured to compute the user's Normal Brain Profile therefrom. After reading this description, numerous computation methods that can be employed for making this calculation will become apparent to those skilled in the art. However, for the purposes of continuing with the description of this example, the particular method used to compute the user's Normal Brain Profile from their earlier-computed Qualified Brain Profile Scores is not critical.

Once the user's Normal Brain Profile has been computed by the computer system, the monitoring of the user's Brain Profile can begin. As such, if the user wishes to immediately initiate the monitoring process, and if the maximum number of Daily Test Sessions has not yet been reached, he or she can do so by merely obtaining a new Test Session ID. However, if the maximum number of daily Test Sessions has been reached, then the user will need to wait for the Test Session Trial Number to reset; access the computer system's server device with the system's personal computing device; and employ the Web-Based User Log-In to obtain a new Test Session ID.

Regardless of when a new Test Session ID is assigned, after the corresponding Test Session has been activated, from this point forward in the process, the answer to the question as to whether the user's Normal Brain Profile has been established will be “YES”. As such, the brain performance data collected from any subsequently-completed Test Session will now be used by the computer system to compute the user's “Current Brain Profile”.

In this example, the same computation system and methodology that was used to compute the user's Normal Brain Profile, is also used to determine their Current Brain Profile. As mentioned above, after reading this description, numerous computation systems and methods for making this calculation will become apparent to those skilled in the art. However, for the purposes of continuing with the description of this example, the particular system and method used to compute the user's Current Brain Profile is not critical.

The Test Session employed in the monitoring phase (i.e., in the phase after the user's Normal Brain Profile has been established) is the same as the one employed in the phase wherein the user's Normal Brain Profile was being established. As such, when the user completes each of that test session's modules, the computer system is programmed and configured to compute the module's Current Test Module Score from the collected brain performance data. After reading this description, numerous computation methods that can be employed for making this calculation will become apparent to those skilled in the art. However, for the purposes of continuing with the description of this example, the particular system and method used to compute a module's Current Test Module Score is not critical.

The phrase “the same”, as it refers to the test session being employed, encompasses test sessions which are completely identical to one another; as well as the following: (a) test sessions where the order of the modules are randomized, but the inquiries therein are identical; (b) test sessions where the modules are in the same order, but the inquiries therein are randomized; and (c) test sessions where both, the order of the modules and the inquiries therein, are randomized. However, for the purposes of continuing with the description of this particular example, whether, and to what extent, the order of the modules and/or the inquiries therein are randomized, is not critical.

After a Current Test Module Score has been computed for each of the test session's modules, the computer system is programmed and configured to compute the user's Current Brain Profile therefrom. After reading this description, numerous computation methods that can be employed for making this calculation will become apparent to those skilled in the art. However, for the purposes of continuing with the description of this example, the particular system and method used to compute the user's Current Brain Profile is not critical.

After the user's Current Brain Profile has been calculated, the computer system is programmed and configured to compute the difference, if any, between the their Current Brain Profile and their earlier-established Normal Brain Profile. While it is possible for these two scores to be identical, since the various test sessions are often taken at different times and under different circumstances, it is highly unlikely for them to be exactly the same. After reading this description, numerous computation methods that can be employed for making this calculation will become apparent to those skilled in the art. However, for the purposes of continuing with the description of this example, the particular system and method used to compute this difference, if any, is not critical.

When practicing this invention, the mere fact that a difference exists between the user's Current Brain Profile and their Normal Brain Profile does not necessarily mean that the user's Brain Profile has changed. For example, as mentioned above, in accordance with this invention, the user's Normal Brain Profile is not a “baseline”; but rather, a “consistent range” of scores. Thus, even if a difference exists, as long as that difference falls within the “consistent range” of their Normal Brain Profile scores, the user's Brain Profile will be deemed to have not changed.

As such, in the embodiment illustrated in FIG. 1, once the computer system computes the difference between the user's Current Brain Profile and their Normal Brain Profile, it is then programmed and configured to compute whether the difference falls within the “consistent range” of that user's earlier-established Normal Brain Profile. After reading this description, numerous computation methods that can be employed for making this calculation will become apparent to those skilled in the art. However, for the purposes of continuing with the description of this example, the particular system and method used to compute whether the difference, if any, falls within or outside of the consistent range of the user's Normal Brain profile is not critical.

If the difference falls within the user's uniquely-specific “consistent range” of their Normal Brain Profile, the computer system is programmed and configured to immediately report: “NO CHANGE in the User's Brain Profile”, or with language or imagery of similar import. However, if the difference falls outside of the user's uniquely-specific “consistent range” of their Normal Brain Profile, the computer system is programmed and configured to immediately report: “CHANGE in the User's Brain Profile”, or with language or imagery of similar import. After reading this description, numerous methods of reporting whether or not there is a change in a user's Brain Profile will become apparent to those skilled in the art. Such methods include, for example, words, pictures, recordings, charts, graphs, colors, lights, etc. However, for the purposes of continuing with the description of this example, the particular reporting system and methodology employed is not critical.

As used in this example, the term “immediate” or “immediately”, as it refers to the speed in which the results are reported to the user, is a relative designation, and does not necessarily mean instantaneously. For example, although computer systems can make fairly complicated computations in a relatively short period of time, there is, none-the-less, a time lag associated therewith. That being said, depending upon the speed of the communications network being employed, as well as upon the processing speed of the particular personal computing device and/or server device making up the computer system, in most instances, the time lag associated with reporting the results to the user will generally range from between 0.1 second to 15 minutes after the test session is completed. However, since an individual, their parent, their guardian, their caregiver, or some other third party would typically like such results as soon as possible, when practicing preferred embodiments of this invention, the computer system and/or communications network should be chosen, programmed, and/or configured such that a report can be generated between 0.1 second to 10 minutes; preferably, from between 0.1 second to 5 minutes; and more preferably, from between 0.1 second to 1 minute after a particular test session is completed. Any of the aforementioned time lags associated with the reporting function of the present invention are deemed to be encompassed by the term “immediate” or “immediately” as used herein.

As mentioned above, in addition to calculating a Brain Profile Score for each test session, in certain embodiments, the computer system can also be programmed and configured to calculate a “normal” score for each test module making up a particular test session—i.e., a user's “Normal Test Module Score”. In such an embodiment, the user's Normal Test Module Scores can be used to calculate the individual's Normal Brain Profile and/or their Current Brain Profile.

One example of a process wherein Normal Test Module Scores are used to calculate one's Normal Brain profile is described below and illustrated in FIG. 2. Upon reading the description of FIG. 2, numerous other suitable methods and systems, which can be employed to determine whether there is a change to a user's Brain Profile, will become apparent to those skilled in the art. Such suitable methods and systems are deemed to be encompassed by the present invention.

In FIG. 2, the illustrated embodiment is essentially the same as that illustrated in FIG. 1, with the exception as to how the user's Normal Brain Profile is computed. Specifically, in FIG. 1, the user's Normal Brain Profile is computed from a mathematical manipulation of their earlier-computed Qualified Brain Profile Scores. However, in FIG. 2, the user's Normal Brain Profile is computed from a mathematical manipulation of their earlier-computed Normal Test Module Scores. The manner in which the FIG. 2 example computes a user's Normal Test Module Scores and, thereafter, their Normal Brain Profile, will now be discussed.

In FIG. 2, the steps employed in the process, up to the point where the computer system makes the determination as to whether the user's Normal Brain Profile has been established, are the same as those for FIG. 1. If the user's Normal Brain Profile has not yet been established, as in FIG. 1, the computer system is programmed and configured to direct the user to take and complete the various Test Modules making up the particular Test Session. Thereafter, also as in FIG. 1, upon the completion of each Test Module, the computer system is programmed and configured to compute a specific Test Module Score based upon that module's collected brain performance data; and to make the determination as to whether the Test Module Score is “Qualifying” or “Non-Qualifying”.

However, unlike FIG. 1, in the FIG. 2 embodiment, the computer system is programmed and configured to calculate a “Normal Test Module Score” for each module from earlier-computed Qualifying Test Module Scores. Thereafter, computer system is programmed and configured to calculate the user's Normal Brain Profile from the compilation of all the session's earlier-computed Normal Test Module Scores.

Specifically, after the user completes the first Test Module, as in FIG. 1, the computer system is programmed and configured to calculate a Test Module Score from that module's collected brain performance data; and to determine whether that Test Module Score is “Qualifying” or “Non-Qualifying”. However, in the FIG. 2 example, after the Test Module Score has been identified as “Qualifying” or “Non-Qualifying”, the computer system is programmed and configured to determine whether there are enough earlier-computed Qualifying Test Module Scores for it to compute the user's Normal Test Module Score for that particular module. After reading this description, numerous computation methods that can be employed for making this determination will become apparent to those skilled in the art. All such methods, be they past, present or future, are deemed to be encompassed by the present invention.

As mentioned earlier, at least two Qualifying Test Module Scores are necessary to compute a particular module's Normal Test Module Score. Since this is the first time that the particular Test Module was completed, at this point in the process, even if the resulting Test Module Score was classified as “Qualifying”, the answer to the question as to whether enough Qualifying Test Module Scores for that module have been collected to calculate its Normal Test Module Score will be “NO”.

As such, the computer system is programmed and configured to then make the determination of whether all of the test modules making up the particular test session have been completed by the user. Since this invention requires that each test session comprises at least two test modules, and since the test module which was just completed was also the first test module, at this point in the process, the answer to the question as to whether all of the session's test modules have been completed by the user will be “NO”.

Accordingly, the computer system is programmed and configured to prompt the user to take and complete the remaining test modules making up the particular test session. As each test module is completed, the computer system is programmed and configured to compute that module's Test Module Score; to determine whether the computed Test Module Score is “Qualifying”; and to make the determination as to whether enough Qualifying Test Module Scores have been collected in order to compute that module's Normal Test Module Score. As before, since a Normal Test Module Score must be calculated from at least two earlier-computed Qualifying Test Module Score, and since this is the first time that any of the session's modules are being completed, at this point in the process, regardless of whether any or all of the Test Module Score were classified as “Qualifying”, the answer to the question as to whether enough Qualifying Test Module Scores for each module has been collected to calculate its respective Normal Test Module Score will be “NO”. As such, in this first pass of the test session, no Normal Test Module Scores can be calculated.

Notwithstanding the foregoing, after all of the test modules from this first pass of the test session have been completed, the computer system is programmed and configured to determine whether a Normal Test Module Score has been computed for each of its test modules. At this point in the process, the answer to this determination will be “NO”. As such, the computer system is programmed and configured to prompt the user to take and complete another test session.

Until the user's Normal Brain Profile has been established, as each module of a subsequent test session is completed, the computer system is programmed and configured to compute that module's Test Module Score; to make the determination as to whether the computed Test Module Score is “Qualifying”; and to make the determination as to whether enough Qualifying Test Module Scores have been collected in order to compute that module's Normal Test Module Score.

Once enough of the user's earlier-computed Qualifying Test Module Scores have been collected for a particular module, the computer system is programmed and configured to compute that module's Normal Test Module Score therefrom. This process continues until a Normal Test Module Score has been computed for each of the test session's modules. After reading this description, numerous computation methods that can be employed to make such calculations and re-calculations will become apparent to those skilled in the art. However, for the purposes of continuing with the description of this example, the particular method used to compute and/or recomputed a module's Normal Test Module Score is not critical

Once a module's Normal Test Module Score has been computed, each subsequent time that module is completed, the answer to the question as to whether enough Test Module Scores for that module have been computed will be “YES”. As such, until a Normal Test Module Score has been computed for each of the test session's other modules, in the preferred embodiment illustrated in FIG. 2, the computer system is programmed and configured to recalculate that module's Normal Test Module Score from any subsequently-collected Qualifying Test Module Scores.

Once a Normal Test Module Score has been computed for each module, the computer system is programmed and configured to compute the user's Normal Brain Profile from the test session's complete set of its module's the earlier-computed Normal Test Module Scores. After reading this description, numerous computation methods that can be employed to make this calculation will become apparent to those skilled in the art. However, for the purposes of continuing with the description of this example, the particular method used to compute the user's Normal Brain Profile Score from the complete set of earlier-computed Normal Test Module Scores is not critical.

As in the FIG. 1 example, in FIG. 2, once the user's Normal Brain Profile has been computed by the computer system, the monitoring of the user's Brain Profile can begin. As such, if the user wishes to immediately initiate the monitoring process, and if the maximum number of daily test sessions has not yet been reached, he or she can do so by merely obtaining a new Test Session ID. However, if the maximum number of daily test sessions has been reached, then the user will need to wait for the Test Session Trial Number to reset; access the computer system's server device with the system's personal computing device; and employ the Web-Based User Log-In to obtain a new Test Session ID.

Regardless of when a new Test Session ID is assigned, after the corresponding test session has been activated, from this point forward in the process, the answer to the question as to whether the user's Normal Brain Profile has been established will be “YES”. As such, the brain performance data collected from any subsequently-completed test sessions will now be used by the computer system to compute the user's “Current Brain Profile”. From this point forward in the FIG. 2 example, the process for determining whether there is a change in the user's Brain Profile, and reporting the same, is the same as that set out in FIG. 1.

As mentioned earlier, the calculation of Normal Test Module Scores for each module can also be employed to monitor how an individual's brain functions are performing by comparing subsequently-calculated Test Module Scores to that same module's earlier-calculated Normal Test Module Score. Moreover, such a comparison can also be employed to calculate the user's Current Brain Profile. One example of such a process is described below and illustrated in FIG. 3. Upon reading the description of FIG. 3, numerous other suitable methods and systems, which can be employed to determine whether there is a change to a user's Brain Profile, will become apparent to those skilled in the art. Such suitable methods and systems are deemed to be encompassed by the present invention.

In FIG. 3, the embodiment is similar to FIG. 2, in that the user's Normal Brain Profile is computed from the complete set of the user's earlier-computed Normal Test Module Scores. Also as in FIG. 2 embodiment, in FIG. 3, once the user's Normal Brain Profile is established, the monitoring process can begin by the subsequent calculation of the user's Current Brain Profile. However, in the FIG. 3 embodiment, the user's Current Brain Profile is computed differently.

Specifically, when calculating the user's Current Brain Profile in the FIG. 3 embodiment, after each of the test session's modules is completed, the computer system computes that module's Current Test Module Score. However, instead of just proceeding to the next test module as in the FIG. 2 embodiment, in FIG. 3, the computer system is programmed and configured to compare that module's Current Test Module Score to its earlier-computed Normal Test Module Score, and to calculate therefrom a “Test Module Differential Score” for that particular module. This process continues until a Test Module Differential Score has been computed for each of the session's modules. After reading this description, numerous computation methods that can be employed to make this calculation will become apparent to those skilled in the art. However, for the purposes of continuing with the description of this example, the particular method used to compute a module's Test Module Differential Score is not critical.

Once a Test Module Differential Score has been calculated for each of the session's modules, the computer system is programmed and configured to compute therefrom the user's Current Brain Profile Score. After reading this description, numerous computation methods that can be employed when practicing this invention will become apparent to those skilled in the art. However, for the purposes of continuing with the description of this example, the particular method used to compute the user's Current Brain Profile from the complete set of earlier-computed Test Module Differential Scores is not critical. From this point forward in the FIG. 3 example, the process for determining whether there is a change in the user's Brain Profile, and reporting the same, is the same as that set out in FIG. 2.

### Test Sessions and Test Modules

While the present invention requires the implementation of at least two separate and distinct test modules within any given test session, it is in no way limited to that number. As mentioned above, if the intent is to establish a fairly comprehensive Brain Profile, the selection, number, and configuration of test modules should be such that they are cumulatively designed to test neuromotor and cognitive brain functions controlled by at least 2; preferably, at least 4; more preferably, at least 6; and even more preferably, at least 8 of the following domains of the human brain: Executive, Processing Speed, Episodic Memory, Echoic Memory, Visual-Spatial, Working Memory, Semantic Memory, Attention, Verbal Fluency, Orientation, and Cerebral. Specific neuromotor and cognitive brain functions controlled by these domains of the human brain are known to those skilled in the art; and, a sampling of them is described supra.

Depending upon which neuromotor and cognitive brain functions the end user wants to monitor, the number and design of test modules making up a particular test session encompassed by the present invention are limitless. That being said, a particular example of a test session which is designed to test and monitor a fairly comprehensive grouping of neuromotor and cognitive brain functions is one that test and monitors at least the following brain functions: visual short and long term recall; auditory short and long term recall; visual perception; visual numerical working memory; eye-hand coordination; manual dexterity; processing speed; movement speed; vigilance; postural stability; situational awareness; ability to suppress well-learned responses; and various neuromotor, cognitive and judgment skills. After reading this description, numerous test modules, test module designs and test module configurations that can be employed when practicing this invention will become apparent to those skilled in the art. All of such modules, designs and configurations are deem to be encompassed by the present invention.

A specific, yet non-limiting, example of a test session configuration is set out below. This particular test session provides for a fairly comprehensive evaluation of many of the brain functions that may be affected by any one or more of the following: brain injuries; brain diseases; psychiatric disorders; aging; fatigue; stress; sleep deprivation; pain; the ingestion, inhalation and/or injection of narcotic or controlled substances; and the consumption of alcoholic or other intoxicating beverages. A detailed explanation of the modules making up this particular illustrative example of a comprehensive test session will now be discussed.

### Long Term Visual Memory Recall Module

This module consists of a “START” designation which is visually displayed on the personal computing device. The subject is instructed to press the “START” designation using a designated input controller on the personal computing device in order to begin the test.

This module also consists of two distinct segments—the “Remember Segment” and the “Recall Segment”. These two segments are separated by at least one additional test module. Preferably, the Remember Segment of this module is towards the beginning of the test session; and, the Recall Segment of this module is towards the end of the test session.

In the Remember Segment, a certain number of images are visually displayed to the subject. The images are randomly selected by the computer system from a library of figures, and presented, one at a time, on the screen of the personal computing device. Each time a figure is presented, the computer system is programmed to only display it on the screen for a relatively short period of time—generally, for less than 2 seconds; and preferably, for less than 1 second. The subject is instructed to remember these images for recall at a later time in the test session.

In the Recall Segment, a certain number of images are again visually displayed, one at a time, to the subject. At least a portion of the images presented in the Recall Segment are the same as those presented in the corresponding Remember Segment of this module. The subject will be provided with a set period of time to use a designated input controller on the personal computing device to identify whether the particular image being displayed was also displayed in the corresponding Remember Segment—generally, less than 5 seconds; and preferably, less than 3 seconds.

The object of this module is for the subject to correctly identify all of those images displayed in the Recall Segment, which were also displayed in the corresponding Remember Segment.

This module collects performance data pertaining to a subject's long-term visual memory recall brain function by measuring how many correct and timely answers were input by the subject during its Recall Segment, as well as how many incorrect or untimely answers were input.

### Long Term Auditory Memory Recall Module

This module consists of a “START” designation which is visually displayed on the personal computing device. The subject is instructed to press the “START” designation using a designated input controller on the personal computing device in order to begin the test.

This module also consists of two distinct segments—the “Remember Segment” and the “Recall Segment”. These two segments are separated by at least one additional test module. Preferably, the Remember Segment of this module is towards the beginning of the test session; and, the Recall Segment of this module is towards the end of the test session.

In the Remember Segment, a certain number of words are verbally presented to the subject. The words are randomly selected by the computer system from a library of spoken words, and presented, one at a time, through the speakers of the personal computing device. The subject is instructed to remember these words for recall at a later time in the test session.

In the Recall Segment, a certain number of words are again verbally presented, one at a time, to the subject. At least a portion of the words presented in the Recall Segment are the same as those presented in the corresponding Remember Segment of this module. The subject will be provided with a set period of time to use a designated input controller on the personal computing device to identify whether the particular word being presented was also presented in the corresponding Remember Segment—generally, less than 5 seconds; and preferably, less than 3 seconds.

The object of this module is for the subject to correctly identify all of those words presented in the Recall Segment, which were also presented in the corresponding Remember Segment.

This module collects performance data pertaining to a subject's long-term auditory memory recall brain function by measuring how many correct and timely answers were input by the subject during its Recall Segment, as well as how many incorrect or untimely answers were input.

### Finger Tapping Module

This module consists of a “START” designation which is visually displayed on the personal computing device. The subject is instructed to press the “START” designation using a designated input controller on the personal computing device in order to begin the test.

The subject will also be instructed that shortly after the “START” designation is pressed, they will need to use one of their fingers (preferably, the index finger of their dominant hand) to tap a designated input controller of the personal computing device (e.g., the space bar, any key on a conventional or screen-displayed keyboard, etc.) as many times as possible within a set period of time.

The object of this module is for the subject to tap the designated input controller with their finger as many times as possible within a set period of time. If desired, prior to scoring the results of this module, the computer system can be programmed to have the subject repeat this test at least one additional time.

This module collects performance data pertaining to those brain functions which control a subject's eye-hand coordination and manual dexterity by measuring how many times the designated input controller was tapped during the test.

### Speed & Accuracy Finger Tapping Module

This module consists of a “START” designation which is visually displayed on the personal computing device. The subject is instructed to press the “START” designation using a designated input controller on the personal computing device in order to begin the test.

The subject will also be instructed that, shortly after the “START” designation is pressed, they will need to use one of their fingers (preferably, the index finger of their dominant hand) to tap at least two separate designated input controllers of the personal computing device (e.g., the space bar, any key on a conventional or screen-displayed keyboard, etc.) as many times as possible within a set period of time. Preferably, the designated input controllers that are to be pressed during this test are spaced relatively far apart from one another.

The object of this module is for the subject to tap the designated input controllers with their finger as many times as possible within a set period of time. If desired, prior to scoring the results of this module, the computer system can be programmed to have the subject repeat this test at least one additional time.

This module collects performance data pertaining to those brain functions which control a subject's eye-hand coordination, visual neuromotor channel capacity, manual dexterity, and coordination by measuring how many times the designated input controllers were tapped, as well as how many times the wrong input controllers were tapped.

### Short-Term Sequential Image Memory Recall Module

This module consists of a “START” designation which is visually displayed on the personal computing device. The subject is instructed to press the “START” designation using a designated input controller on the personal computing device in order to begin the test.

This module also consists of two distinct segments—the “Remember Segment” and the “Recall Segment”. Unlike some of the earlier-described modules where the Remember and Recall Segments are separated by other modules, in this Module, both segments are completed in the same test.

In the Remember Segment, a certain number of images are visually displayed to the subject. The images are randomly selected by the computer system from a library of figures, and presented, one at a time, on the screen of the personal computing device. Each time a figure is presented, the computer system is programmed to only display it on the screen for a relatively short period of time—generally, for less than 2 seconds; and preferably, for less than 1 second. The subject is instructed to remember each of these images, and the order in which they were displayed.

In the Recall Segment, all of the images displayed in Remember Segment are displayed at the same time on the screen of the personal computing device. If desired, additional images, which were not shown to the subject in the Remember Segment, can also be displayed at this time. The subject is then instructed to use a designated input controller on the personal computing device to identify only those images displayed in the corresponding Remember Segment, as well as the order in which they were displayed.

The subject will have a set time to complete this task—generally less than 1 minute; and preferably, less than 30 seconds. After the second Recall Segment is completed, this test module can end, or the computer system can be programmed to proceed with subsequent Remember and Recall Segments configured as above.

The object of this module is for the subject to remember which images had their color changed, and in what order it happened.

This module collects performance data pertaining to a subject's short-term sequential memory recall brain function by measuring how many correct and timely answers were input by the subject during its Recall Segment, as well as how many incorrect or untimely answers were input. Note, if the Recall Segment of this module also displays images which were not displayed in the corresponding Remember Segment, this module can also collect performance data pertaining to a subject's short-term visual memory recall brain function.

### Short-Term Sequential Spatial Visual Working Memory Module

This module consists of a “START” designation which is visually displayed on the personal computing device. The subject is instructed to press the “START” designation using a designated input controller on the personal computing device in order to begin the test.

This module also consists of two distinct segments—the “Remember Segment” and the “Recall Segment”. Unlike some of the earlier-described modules where the Remember and Recall Segments are separated by other modules, in this module, both segments are completed in the same test. Also, unlike some of the earlier-described modules, the present module consists of multiple Remember Segments and multiple Recall segments.

Prior to the initiation of any Remember or Recall Segment, the program visually displays the same, but multiple, images on the screen of the personal computing device. These images will remain stationary on the screen throughout the test. Preferably, there are at least 4 images; more preferably, at least 6; and even more preferably, at least 8 images being displayed. At the beginning of this test, all images are the same color. However, during the test, the color of certain images will change, one at a time, from a first color, to a second color, and then back to the first color. If a figure's color is changed, the second color will remain displayed on the screen for only a relatively short period of time—generally, for less than 2 seconds; and preferably, for less than 1 second.

In the first Remember Segment of this module, the computer system randomly changes the color of some of the displayed images, one at a time, and in a set order. The subject is instructed to remember each of the images which had their color changed in this Remember Segment, and the order in which that happened.

In the first Recall Segment of this module, the program reverts all of the images back to their first color. Thereafter, the subject is instructed to use a designated input controller on the personal computing device to identify only those images whose color was changed, as well as in what order that happened. After the first Recall Segment is completed, the program proceeds to the second Remember Segment.

In the second Remember Segment, the computer system will begin by changing the color of those images that were changed in the immediately preceding (i.e., in this case, the first) Remember Segment; and, changing them in the exact same order. Thereafter, the computer system will randomly change the color of at least one (preferably, more than one) additional image. As before, the subject is instructed to remember each image which had their color changed in this Remember Segment, and the order in which that happened.

In the second Recall Segment, the program reverts all of the images back to their first color. Thereafter, the subject is instructed to use a designated input controller on the personal computing device to identify only those images whose color was changed in the corresponding Remember Segment, as well as in what order that happened. After the second Recall Segment is completed, this test module can end, or the computer system can be programmed to proceed with subsequent Remember and Recall Segments configured as above.

The object of this module is for the subject to remember which images had their color changed, and in what order it happened.

This module collects performance data pertaining to a subject's short-term sequential spatial working memory brain function by measuring how many correct and timely answers were input by the subject during its various Recall Segments, as well as how many incorrect or untimely answers were input.

### Short-Term Sequential Visual Numerical Working Memory Module

This module consists of a “START” designation which is visually displayed on the personal computing device. The subject is instructed to press the “START” designation using a designated input controller on the personal computing device in order to begin the test.

This module also consists of two distinct segments—the “Remember Segment” and the “Recall Segment”. Unlike some of the earlier-described modules where the Remember and Recall Segments are separated by other modules, in this module, both segments are completed in the same test. Also, unlike some of the earlier-described modules, the present module consists of multiple Remember Segments and multiple Recall segments.

In the first Remember Segment, a certain set of numbers selected from “0” to “9” are visually displayed to the subject, one at a time, on the screen of the personal computing device. The numbers are randomly selected by the computer system. The numbers can be repeated and presented in any order. Each time a number is presented, the computer system is programmed to only display it on the screen for a relatively short period of time—generally, for less than 2 seconds; and preferably, for less than 1 second. The subject is instructed to remember each of these numbers, and the order in which they were displayed.

In the first Recall Segment, the program visually displays on the screen of the personal computing device an image similar to a key pad of a conventional touch tone telephone. The subject is then instructed to use a designated input controller on the personal computing device to identify only those numbers that were displayed in the corresponding Remember Segment, as well as the order in which they were displayed. The subject will have a set time to complete this task—generally, less than 1 minute; and preferably, less than 30 seconds. After the first Recall Segment is completed, the program proceeds to the second Remember Segment.

In the second Remember Segment, the program again visually and individually displays to the subject a certain numbers selected from “0” to “9”. The display of this Remember Segment begins with the same exact numbers and the same order as in the immediately preceding (i.e., in this case, the first) Remember Segment. Thereafter, the computer system will randomly select and display at least one (preferably, more than one) additional number. As before, the subject is instructed to remember each of the numbers that were displayed in this Remember Segment, and the order in which they were displayed.

In the second Recall Segment, the program again visually displays the telephone key pad image. The subject is then instructed to use a designated input controller on the personal computing device to identify only those numbers that were displayed in the corresponding Remember Segment, as well as the order in which they were displayed. The subject will have a set time to complete this task—generally, less than 1 minute; and preferably, less than 30 seconds. After the second Recall Segment is completed, this test module can end, or the computer system can be programmed to proceed with subsequent Remember and Recall Segments configured as above.

The object of this module is for the subject to remember which images had their color changed, and in what order it happened.

This module collects performance data pertaining to a subject's short-term sequential visual numerical working memory brain function by measuring how many correct and timely answers were input by the subject during its various Recall Segments, as well as how many incorrect or untimely answers were input.

### Single Choice Processing Speed, Movement Speed & Vigilance Module

This module consists of a “START” designation which is visually displayed on the personal computing device. The subject is instructed to press the “START” designation using a designated input controller on the personal computing device in order to begin the test.

This module also consists of three solitary images which are visually-displayed at the same time on the personal computing device. The first image is a stationary figure which remains unchanged. This first image represents a “home base”, and is present on the personal computing devices' screen throughout the test.

The second image is also stationary, spatially displaced from the first image, and present on the personal computing devices' screen throughout the test. This second image has its color randomly change from a first color to a second color. This random changing of the second image's color is performed by computer system.

The third image is a single figure which is movable and controlled by the subject using a designated input controller of the personal computing device. This third image is present on the personal computing devices' screen throughout the test.

In this test, the program displays all of the three images on the screen of the personal computing device at the same time. Thereafter, the subject is instructed to use the designated input controller to move the third image onto the first image. After the third image has been moved onto the first image, the computer system will begin to randomly change the color of the second image from its first floor to its second color. The subject is instructed to observe when the second image has its color changed, and to immediately thereafter, move the third image to the second image, and then back to the first image. As soon as the third image has moved onto the second image, the computer system reverts that second image's color back to its first color. Once the third image has been moved back onto the first image, the designation process will continue by the computer system again randomly changing the color of the second image. The computer system is programmed to perform this designation process a set number of times during the test.

The object of this module is for the subject to move the third image from the home base (i.e., the first image) to the second image only when its color has been changed, and then back to the home base, as quickly and as accurately as possible, within a set period of time.

This module collects performance data pertaining to a subject's visual information processing speed, movement speed and vigilance brain functions by measuring reaction time, movement time, and response time.

### Multiple Choice Processing Speed, Movement Speed & Vigilance Module

This module consists of a “START” designation which is visually displayed on the personal computing device. The subject is instructed to press the “START” designation using a designated input controller on the personal computing device in order to begin the test.

This module also consists of three classes of images which are visually displayed at the same time on screen of the personal computing device. The first class consists of a single stationary figure which remains unchanged. This first image represents a “home base”, and is present on the personal computing devices' screen throughout the test.

The second class is a grouping of multiple figures which are also stationary, spatially displaced from the first image, and present on the personal computing devices' screen throughout the test. Each of the figures in this second class has its color randomly changed from a first color to a second color. This random changing of the second class of image's color is performed by computer system.

The third image is a single figure which is movable and controlled by the subject using a designated input controller of the personal computing device. This third image is present on the personal computing devices' screen throughout the test.

In this test, the program displays all of the three classes of images on the screen of the personal computing device at the same time. Thereafter, the subject is instructed to use the designated input controller to move the third image onto the first image. After the third image has been moved onto the first image, the computer system will begin to randomly change the color of certain of the second images from its first color to its second color, one at a time. The subject is instructed to observe which of the second class of images has their color changed, and to immediately thereafter, move the third image to that particular second image, and then back to the first image. As soon as the third image has moved onto the second image whose color has changed, the computer system reverts that second image's color back to its first color. Once the third image has been moved back onto the first image, the designation process will continue by the computer system again randomly changing the color of one of the figures in the second class of images. Note, since the selection is random, it is possible for the subsequent color change to be the same as the immediately preceding one. The computer system is programmed to perform this designation process a set number of times during the test.

The object of this module is for the subject to move the third image from the home base (i.e., the first image) to that particular image from the second class whose color changed, and then back to the home base, as quickly and as accurately as possible, within a set period of time.

This module collects performance data pertaining to a subject's visual information processing speed, movement speed and vigilance brain functions by measuring reaction time, movement time, and response time.

### Situational Awareness Module

This module consists of a “START” designation which is visually displayed on the personal computing device. The subject is instructed to press the “START” designation using a designated input controller on the personal computing device in order to begin the test.

This module also consists of six images which are visually displayed on the screen of the personal computing device. The first image is a single stationary figure which remains unchanged. This figure represents a “home base” and is present on the personal computing devices' screen throughout the test.

The second image is a single figure which is spatially displaced from the first image. The computer system randomly displays this second image on the personal computing devices' screen at various times throughout the test. When displayed, the image remains on the screen for only a relatively short period of time—generally, generally, for less than 2 seconds; and preferably, for less than 1 second.

The third image is a single figure which is spatially displaced from the first image. The computer system randomly displays this third image on the personal computing devices' screen at various times throughout the test. When displayed, the image remains on the screen for only a relatively short period of time—generally, generally, for less than 2 seconds; and preferably, for less than 1 second. This third image is identical to the second image, with the only difference being its color.

The fourth image is a single figure which is spatially displaced from the first image. The computer system randomly displays this fourth image on the personal computing devices' screen at various times throughout the test. When displayed, the image remains on the screen for only a relatively short period of time—generally, generally, for less than 2 seconds; and preferably, for less than 1 second When displayed, the image remains on the screen for only a relatively short period of time—generally, generally, for less than 2 seconds; and preferably, for less than 1 second. This fourth image is different from any of the other images; but, has the same color as the second image.

The fifth image is a single figure which is spatially displaced from the first image. The computer system randomly displays this fifth image on the personal computing devices' screen at various times throughout the test. When displayed, the image remains on the screen for only a relatively short period of time—generally, generally, for less than 2 seconds; and preferably, for less than 1 second. This fifth image is identical to the fourth image, with the only difference being its color. The color of the fifth image is the same color as that of the third image.

The sixth image is a single figure which is movable and controlled by the subject using a designated input controller of the personal computing device. This image is present on the personal computing devices' screen throughout the test.

In this test, the subject is instructed to remember the shape and color of only one of the second through fifth images—hereinafter, referred to as the “designated image”. The computer system is programmed to display the first and sixth image on the screen of the personal computing device at the same time. As mentioned above, these two images will remain on the screen throughout the test. Thereafter, the subject is instructed to use the designated input controller to move the sixth image onto the first image.

After the third image has been moved onto the first image, the computer system will begin to periodically and randomly display on the personal computing device's screen one of images two through five, one at a time. Each time one of the two through five images are displayed, they only remain on the screen for a relatively short period of time—generally, for less than 2 seconds; and preferably, for less than 1 second.

The subject is instructed to observe the second through fifth images as they are displayed on the screen to see when the “designated image” appears. The subject is also instructed that, immediately upon seeing the designated image displayed, they are to move the sixth image to the designated image, and then back to the first image. Once the sixth image has been moved back onto the first image, the designation process will continue by the computer system again randomly displaying, one at a time, one of images two through five. The computer system is programmed to perform this designation process a set number of times during the test.

The object of this module is for the subject to move the sixth image from the home base (i.e., the first image) to the designate image when it appears, and then back to the home base, as quickly and as accurately as possible, within a set period of time.

This module collects performance data pertaining to a subject's visual information processing speed, movement speed and vigilance brain functions by measuring reaction time, movement time, and response time, as well as the subject's ability to suppress well learned responses.

### Integrated Neuromotor, Cognitive & Judgment Skill Module—Attraction Test

This module consists of a “START” designation which is visually displayed on the personal computing device. The subject is instructed to press the “START” designation using a designated input controller on the personal computing device in order to begin the test.

This module also consists of two classes of images which are visually-displayed on the personal computing device. The first class consists of a solitary figure whose linear horizontal movement is controlled by the subject using a designated input controller of the personal computing device. This image is present on the lower portion of the personal computing devices' screen throughout the test.

The second class consists of multiple figures whose random vertical linear movements, from the upper portion to the lower portion of the screen of the personal computing device, is controlled by the computer system. The size and shape of all of the figures in the second class of images are such that they can completely fit into the first image.

In this test, the computer system displays multiple figures from this second class randomly moving at the same time from the upper portion to the lower portion of the screen of the personal computing device throughout the test. The computer system also randomly controls the horizontal points across the upper portion of the personal computing device's screen from which second class of images originate prior to their vertical movement towards the lower portion of the screen, as well as the speed at which these images move. The subject is instructed that, before any of the individual figures of the second class of images reaches the lower portion of the screen, the first image should be moved horizontally such that the individual figures of the second class of images fit within the first image, without hitting any of the first image's side walls.

The object of this module is for the subject to move the first image in such a manner to have as many of the individual figures of the second class of images fit within the first image, without hitting any of the first image's side walls, within the set period of time.

This module collects performance data pertaining to those brain functions which control a subject's neuromotor, cognitive and judgment skills by measuring how many individual figures of the second class of images were able to completely fit into the first image; as well as, how many times the individual figures of the second class of images did not completely fit into, but rather hit and bounced off of, the side walls of the first image.

### Integrated Neuromotor, Cognitive & Judgment Skill Module—Maze Test

This module consists of a “START” designation which is visually displayed on the personal computing device. The subject is instructed to press the “START” designation using a designated input controller on the personal computing device in order to begin the test.

This module also consists of one movable image and one stationary maze through which the image can pass—both of which are visually displayed on the personal computing device at the same time throughout the test. While continuous, the maze is divided up into segments so as to be able for the computer system to collect data as to how much of the maze was completed within a set period of time. The image's movement is controlled by the subject using a designated input controller of the personal computing device.

In this test, the subject is instructed to use a designated input device of the personal computing system to move the movable image through as much of the maze as possible in a set period of time. The subject is also instructed to avoid having the movable image contact the walls of the maze.

The object of this module is for the subject to move the image through the maze as quickly as possible, without hitting the walls of the maze, within a set period of time.

This module collects performance data pertaining to those brain functions which control a subject's neuromotor, cognitive and judgment skills by measuring how much of the maze was completed, how quickly the user moved the image through the maze, and how many times the image hit the maze's walls.

### Integrated Neuromotor, Cognitive & Judgment Skill Module—Avoidance Test

This module consists of a “START” designation which is visually displayed on the personal computing device. The subject is instructed to press the “START” designation using a designated input controller on the personal computing device in order to begin the test.

This module also consists of two classes of images which are visually-displayed on the personal computing device. The first class consists of a solitary figure whose movement is controlled by the subject using a designated input controller of the personal computing device. This image is present on the personal computing device's screen throughout the test.

The second class includes multiple figures whose random speed and random horizontal linear movements from the left portion to the right portion of the screen and from the right portion to the left portion of the screen, are controlled by the computer system. The second class also includes multiple figures whose random speed and random vertical linear from the upper portion to the lower portion of the screen and from the lower portion to the upper portion of the screen, are also controlled by the computer system.

In this test, the computer system displays multiple figures from this second class randomly moving from the upper portion to the lower portion of the screen, from the lower portion to the upper portion of the screen, from the left portion to the right portion of the screen, as well as from the right portion to the left portion of the screen, all at the same time throughout the test. The computer system also randomly controls the horizontal points across the upper and lower portions, as well as the vertical portions along the right and left portions of screen from which second class of images originate prior to their movement. The subject is instructed to move the first image using the designated input controller so as to avoid having that image hit by any of the second images.

The object of this module is for the subject to move the first image in such a manner to avoid getting hit by any of the second images as they move across the screen, within the set period of time.

This module collects performance data pertaining to those brain functions which control a subject's neuromotor, cognitive and judgment skills by measuring how many times the first image was hit by any of the second images during the test.

### Balance & Stability Module

This module consists of a “START” designation which is visually displayed on the personal computing device. The subject is instructed to press the “START” designation using a designated input controller on the personal computing device in order to begin the test.

The subject will also be instructed that, shortly after the “START” designation is pressed, they will need to cross their arms on their chest and begin a process where they stand up and then sit down a set number of times; and, each time they sit down; they are to press a designated input controller device on the personal computing system. After the input controller is pressed, the subject is to re-cross their arm and continues with the standing and sitting process until the set number of trials for the test has been achieved.

The object of this module is for the subject to sit, stand, sit, and press the designated input controller the set number of times, as quickly and accurately as possible.

This module collects performance data pertaining to those brain functions which control a subject's postural stability, eye-hand coordination and manual dexterity by measuring how quickly the number of sit-stand-sit process were completed, as well as how accurately the user press the correct designated input controller at the end of each sit-stand-sit process during the test.

### Order of Test Modules:

The order in which the modules of the aforementioned illustrative test session is important. It is also preferred for this embodiment for the order of these particular modules not to be randomized in subsequent test sessions taken by the subject. That being said, as mentioned earlier, it is within the scope of this invention for the order of the modules to be randomized. If randomization is desired, while the randomization can consist of a complete randomization of all modules, in a preferred embodiment, randomization is limited to test modules within the same grouping.

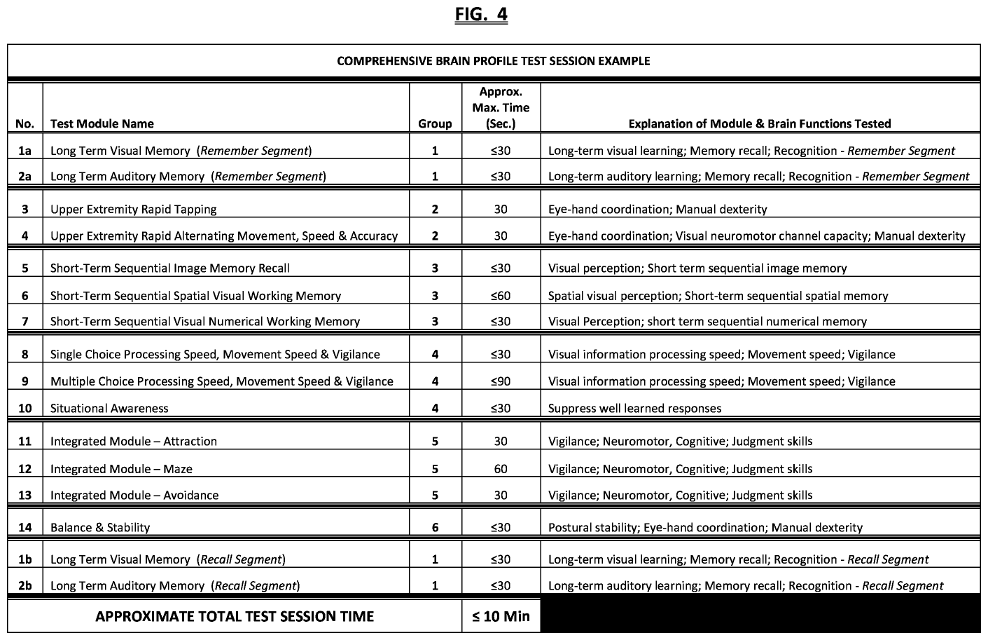

An example of how the modules described above can be grouped is illustrated in FIG. 4. Specifically, in FIG. 4, the Long Term Visual Memory Recall Module and the Long Term Auditory Memory Recall Module are deemed to be in the same group (hereinafter, “Module Group 1”). As such, while these modules should be presented one after another in a particular test session, if desired, they can be presented in a randomized order.

Similarly, the Finger Tapping Module and the Speed & Accuracy Finger Tapping Module are deemed to be in the same group (hereinafter, “Module Group 2”). As such, while these modules should be presented one after another in a particular test session, if desired, they can be presented in a randomized order.

Moreover, the Short-Term Sequential Image Memory Recall Module, the Short-Term Sequential Spatial Visual Working Memory Module, and the Short-Term Sequential Visual Numerical Working Memory Module are all deemed to be in the same group (hereinafter, “Module Group 3”). As such, while these modules should be presented one after another in a particular test session, if desired, they can be presented in a randomized order.

Furthermore, the Single Choice Processing Speed, Movement Speed & Vigilance Module, the Multiple Choice Processing Speed, Movement Speed & Vigilance Module, and the Situational Awareness Module are all deemed to be in the same group (hereinafter, “Module Group 4”). As such, while these modules should be presented one after another in a particular test session, if desired, they can be presented in a randomized order.

Next, the Integrated Neuromotor, Cognitive & Judgment Skill Module—Attraction Test, the Integrated Neuromotor, Cognitive & Judgment Skill Module—Maze Test, and the Integrated Neuromotor, Cognitive & Judgment Skill Module—Avoidance Test are all deemed to be in the same group (hereinafter, “Module Group 5”). As such, while these modules should be presented one after another in a particular test session, if desired, they can be presented in a randomized order.

Finally, the Balance & Stability Module is deemed to be in a group all to itself (hereinafter, “Module Group 6”).

In this particular embodiment of a test session, the grouping of modules is preferably presented in the order of Module Group 1 through Module Group 6. Also, if additional modules are added to this test session, each module should be placed in the Module Group to which it most closely relates.

### Additional Test Modules

In addition to the exemplary modules set out above, test sessions encompassed by the present invention may further comprise other modules that do not test or monitor the subject's brain performance data. Examples of such other modules include, without limitation, the following: a registration module, a health & wellness questionnaire module, and various forms and levels of scoring modules. Illustrative examples of such other modules are set out below.

### Registration Modules

In order for the particular subject to register to activate and take a test session, and for that subject's brain performance data to be collected and analyzed, the subject needs to register certain identifying information with the computer system's server. This can be achieved through the implementation of a Registration Module.

If a Registration Module is employed, when the subject connects to the server via a communications network, the subject will be requested to register with the server by inputting certain personal data. A non-limiting example of the type of personal data that may be sought in a Registration Module includes at least the following: the subject's full name, and the subject's full mailing address. In addition to the foregoing, the Registration Module may also request that the subject input at least one of the following additional pieces of personal information: e-mail address(es), phone number(s), date of birth, gender, height, weight, age, gender, and handedness (right, left, ambidextrous). The specific personal information sought to be collected in a Registration Module is dependent largely upon the desired needs and expectations of the end user.

### Health & Wellness Questionnaire Modules

Although the present invention establishes and monitors a subject's Brain Profile, in certain instances, it may be helpful to collect the subject's pertinent health and injury information and history. The collection of such data may be able to assist in the interpretation of the test session scores, as well as the subject's particular Brain Profile. If such information is deemed to be needed, this can be achieved through the implementation of a Health & Wellness Questioner Module.

If a Health & Wellness Questioner Module is employed, the subject will be requested to input certain specific information regarding the same. A non-limiting example of the type of personal data that may be sought in a Health & Wellness Questioner Module includes the following: whether the subject has any history of brain diseases, whether the subject has any history of brain injuries (if so, what, when, and current status and treatment), whether the subject has any history of psychiatric disorders (if so, what, when, and current status and treatment), whether the subject has any history of addictions (if so, what, when, and current status and treatment), whether the subject regularly consumes alcohol (if so, what and how often), whether the subject is taking prescribed or over the counter medication (if so, what and how often), whether the subject regularly consumes caffeine-containing products (if so, what and how often), whether the subject participates in a contact sport (if so, what and how often), whether the subject participates in a physically-demanding profession (if so, what and how often), and/or whether the subject participates in a mentally-demanding profession (if so, what and how often). The specific personal information sought to be collected in a Health & Wellness Questioner Module is dependent largely upon the desired needs and expectations of the end user.

### Scoring Modules

In order for the subject, their parent, guardian or caregiver, or any other party reviewing the results of the tests encompassed by the present invention, once calculated, the results need to be reported. This can be achieved through the implementation of Scoring Modules.

It is within the scope of this invention for there to be various and different Scoring Modules for any given subject completing a test session. For example, one Scoring Module can be reported back to the subject or their parent, guardian or caregiver. Another Scoring Module for the same test session can be reported to the subject's physician. Yet another Scoring Module can be reported to the subject's employer. While all of these Scoring Modules are based upon the same brain performance data collected from the subject in a particular test session, their content and level of detail can vary greatly. The specific information included in a particular Scoring Module is dependent largely upon the following: (a) the particular device's functional capabilities, (b) the end-user's specific needs and requirements, and (c) the current state of the medical profession and/or technology at the time the invention is being practiced.

### Examples of Various Components Associated with the Present Invention

Set out below are non-limiting examples of various components associated with the present invention. Upon reading this description, and as technology advances, those skilled in the art will be able to develop and/or identify additional examples of these components which can be employed. Such additional examples of components are deemed to be encompassed by the present invention.

### Computer System's Personal Computing Device

The personal computing device employed when practicing certain embodiments of this invention can be any such past, present or future device, which can be programmed to communicate, through a communications network (examples defined infra), with a computer server (examples defined infra). Generally, the personal computing device employed when practicing these embodiments of the invention is one that can be configured and programmed to perform at least the following tasks: (a) to communicate with the computer system's computer server via a communications network, (b) to receive and interpret specific information from the system's computer server necessary to activate a test session, (c) to visually display an activated test session to the subject, (d) to receive and interpret brain performance data input from a subject taking the activated test session, (e) to collect and store the received brain performance data input, and (f) to transmit the collected and stored brain performance data input to the system's computer server via a communications network.

In addition to the foregoing, it is within the scope of this invention for the personal computing device to be configured and programmed to do a number of additional tasks and computations, as well as to transmit data to, and/or receive data from, computing devices other than the system's server. Any additional configuration and programming of the personal computing device can be a function of parameters such as: (a) the particular device's functional capabilities, (b) the end user's specific needs and requirements, and (c) the current state of the medical profession and/or technology at the time the invention is being practiced.

The functions that a personal computing device can be programmed to perform are limitless, as are the desired end uses of the information provided when practicing this invention. Accordingly, since computing and communication technologies are constantly changing—as is the understanding of addictions, injuries, diseases, and ailments of the brain—in addition to the foregoing embodiments, the ways in which the personal computing device can be programmed, and still fall within the scope of this invention, are also limitless.

In addition to being configured and programmed to receive and transmit data and information via a communications network, personal computing devices employed when practicing certain embodiments of this invention must also be configured and programmed to display an activated test session to a subject, to receive and interpret brain performance data input from a subject taking the activated test session, and to collect the received brain performance data input. As for the device's ability to display an activated test session, this includes its ability to provide at least a visual display (e.g., through the use of a screen, projection device, etc.).

The device's ability to display an activated test session can also include its ability to provide, among others, at least one of the following: an audio display (e.g., through the use of a speaker), and a physical display (e.g., through the use of a vibrating device). The manner in which an activated test session is displayed to the user is a function of parameters such as: the particular device's functional capabilities, the end-user specific needs and requirements, and the current state of the medical profession and/or technology at the time the invention is being practiced.

That being said, in one presently preferred embodiment, the personal computing device is configured and programmed to display an activated test session by means of a visual display. In another preferred embodiment, the personal computing device is configured and programmed to display an activated test session by means of a visual display and an audio display.

As for the device's ability to receive brain performance data from a user taking an activated test session, this includes its ability to receive user inputs from, among others, at least one or more of the following: a touch screen, a touch pad (e.g., internal, wired, wireless, etc.), a keyboard (e.g., internal, wired, wireless, etc.), a microphone (e.g., internal, wired, wireless, etc.), a camera (e.g., internal, wired, wireless, etc.), a gyroscopic device (e.g., internal, wired, wireless, etc.), and a mouse (e.g., wired, wireless, etc.). The manner in which the device receives brain performance data from a user taking an activated test session is a function of parameters such as: the particular device's functional capabilities, the end-user specific needs and requirements, and the current state of the medical profession and/or technology at the time the invention is being practiced.

That being said, in one presently preferred embodiment, the personal computing device is configured and programmed to receive brain performance data from a user taking an activated test session by means of a mouse (wired or wireless). In another preferred embodiment, the personal computing device is configured and programmed receive brain performance data from a user taking an activated test session by means of a mouse (wired or wireless) and keyboard (internal, wired or wireless). In still another preferred embodiment, the personal computing device is configured and programmed to receive brain performance data from a user taking an activated test session by means of a mouse (wired or wireless), a keyboard (internal, wired or wireless), and a camera (internal, wired, or wireless). In yet another preferred embodiment, the personal computing device is configured and programmed to receive brain performance data from a user taking an activated test session by means of a touch screen. In still a further preferred embodiment, the personal computing device is configured and programmed to receive brain performance data from a user taking an activated test session by means of a touch screen and a gyroscopic device (internal, wired or wireless).

As for the device's ability to collect and store the received brain performance data from an activated test session, this includes its ability to collect and store the data by, among others, at least one or more of the following: primary memory sources (e.g., RAM, DRAM, SRAM, cache, etc.), secondary memory sources (e.g., as tape, magnetic disks, flash drives, optical discs, etc.), and virtual memory sources. The manner in which the device collects and stores brain performance data from a subject taking an activated test session is a function of parameters such as: the particular device's functional capabilities, the end-user specific needs and requirements, and the current state of the medical profession and/or technology at the time the invention is being practiced.

That being said, in one presently preferred embodiment, the personal computing device is configured and programmed to collect and store brain performance data from a user taking an activated test session by means of the device's primary memory. In another preferred embodiment, the personal computing device is configured and programmed to collect and store brain performance data from a user taking an activated test session by means of the device's primary memory and its secondary memory. In still another preferred embodiment, the personal computing device is configured and programmed to collect and store brain performance data from a user taking an activated test session by means of the device's primary memory and its virtual memory.

While there are many different types of personal computing devices that can be used when practicing certain embodiments of this invention, the personal computing devices that currently exist, and that are particularly suitable for such a use, include, without limitation, the following: personal desktop computers, personal laptop computers, personal notebook computers, personal netbook computers, personal hand-held computing note pads, personal hand-held computing tablets, and cellular communication devices. As technology advances, it is envisioned that new personal computing devices will be developed. As long as these new computing devices retain the minimum required features and capabilities of the personal computing devices disclosed herein, the use of such new personal computing devices is encompassed by this present invention.

### Computer System's Server Device

The server device employed when practicing this invention can be any such past, present or future server which can be programmed to communicate, through a communications network (examples defined infra), with the personal computing device. In a particular embodiment, the computer server employed when practicing this invention is one that can be programmed to perform at least the following tasks: (a) to collect and process a subject's specific identification information, (b) to collect and process brain function performance data transmitted thereto from the personal computing device through, or upon which, a test session is taken, (c) computing a test session score through a mathematical manipulation of the brain function performance data, (d) computing a subject's normal Brain Profile score through a mathematical manipulation of a plurality of the subject's previously completed test session scores, (e) through mathematical computations, computing the difference between a subject's specific test session scores and a subject's normal Brain Profile score, and (f) through a mathematical manipulation of the difference between a subject's specific test session scores and a subject's normal Brain Profile, computing whether a subject's specific test session scores constitutes a change to that subject's normal Brain Profile.

In another embodiment, the computer system's server device employed when practicing this invention is programmed to store the calculated results, and to use the stored results, when making subsequent calculations. As such, the subject's normal Brain Profile becomes a dynamic measurement that changes over a period of time as the subject's measured brain performance scores improve, or become impaired.

In yet another embodiment, the computer system's server device employed when practicing this invention is configured and programmed to compute varying levels of changes to a subject's normal Brain Profile. For example, in addition to being programmed to compute whether there is a change in a subject's normal Brain Profile, through a mathematical computation of specific calculated changes and predefined filters which identify acceptable and unacceptable levels of changes, in this particular embodiment, the server is also programmed to computer whether the specific calculated change falls within, or outside of, a predefined acceptable range.

In still a further embodiment, the computer system's server device employed when practicing this invention is configured and programmed to transmit, through a communications network, the subject's specific Brain Profile information. It is within the scope of this particular embodiment of the invention for the server to be programmed to transmit the subject's specific Brain Profile information back to the subject's personal computing device, and/or to any other device which is capable of receiving and processing such a transmission.

While there are many different types of server devices that can be used when practicing certain embodiments of this invention, the server devices that currently exist, and that are particularly suitable for such a use, include, without limitation, the following: local, remote, cloud-based, etc. As technology advances, it is envisioned that new types of server devices will be developed. As long as these new server devices retain the minimum required features and capabilities of the servers disclosed herein, the use of such new server devices is encompassed by this present invention.

The functions that a computer system's sever device can be programmed to perform are limitless, as are the desired end uses of the information provided when practicing this invention. Accordingly, since computing and communication technologies are constantly changing, as is the understanding of certain addictions, injuries, diseases and ailments of the brain, in addition to the foregoing exemplary embodiments, the ways in which the computer system's sever device can be configured and programmed, and still fall within the scope of this invention, are also limitless. This will readily become be apparent to those skilled in the art upon reading this description of the present invention.

### Communications Networks

The communications network employed when practicing this invention can be any such past, present or future communications network over which signals representative of data or instructions can be transmitted via a signal transmission medium. In a particular embodiment, the communications network employed when practicing this invention is one that can be configured to permit, among other things the transmission of signals representative of data or instructions via signal transmitting medium between the a computer system's server and a computer system's personal computing device.

While there are many different types of communications networks that can be used when practicing certain embodiments of this invention, examples of communications networks that currently exist, and that are particularly suitable for such a use, include, without limitation, the following: the internet, an intranet, a wide area networks (WAN), a local area networks (LAN), a virtual private networks (VPN), a cellular network, a satellite network, a cable network, a telephone network, an optical fiber network, etc. As technology advances, it is envisioned that new types of communications networks will be developed. As long as these new communications networks retain the minimum required features and capabilities of the communications networks disclosed herein, the use of such new communications networks is encompassed by this present invention.

The functions that a communications networks can be configured to perform are limitless, as are the desired end uses of the information provided when practicing this invention. Accordingly, since computing and communication technologies are constantly changing, as is the understanding of certain addictions, injuries, diseases and ailments of the brain, in addition to the foregoing exemplary embodiments, the ways in which the communications networks can be configured, and still fall within the scope of this invention, are also limitless. This will readily become be apparent to those skilled in the art upon reading this description of the present invention.

### Computations

The computation method(s) being employed when practicing this invention can be the same for everything (i.e., for modules, for sessions, and for profiles), or there can be a different computation for each type of score being calculated. The particular computation method(s) being chosen will depend, in part, upon the following: (a) the particular functional capabilities of the computer system's various components, (b) the particular functional capabilities of the communications network being employed, (c) the end-user's specific needs and requirements, and (d) the current state of the technology at the time the invention is being practiced. Notwithstanding any of the foregoing, all such computation methods that currently exist, or that are developed in the future, are deemed to be encompassed by this present invention.

As will be apparent to those skilled in the art after reading this description, that there are numerous methods which can be employed to compute the following values: Test Module Scores, Qualifying and Non-Qualifying Test Module Scores, Normal Test Module Scores, Brain Profile Scores, Qualifying and Non-Qualifying Brain Profile Scores, Normal Brain Profile Scores, Current Test Module Scores, Test Module Differentials, Current Brain Profile Scores, differences between Normal Brain Profile Scores and Current Brain Profile Scores, and whether differences between Normal Brain Profile Scores and Current Brain Profile Scores constitutes a change in the individual's Brain Profile. All such computation methods, be they past, present of future, are deemed to be encompassed by the present invention.

### Conclusion

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained. As various changes could be made in the above methods, systems, and mediums without departing from the scope of the invention, it is intended that all matter contained in the above description and shown in the accompanying drawing(s) shall be interpreted as an illustrative, and not in a limiting, sense.

Having described the invention in detail, it will be apparent that modifications and variations are possible without departing from the scope of the invention defined in the appended claims that follow.

1. A method for identifying, monitoring, and reporting, on a computer system, whether a change to the Brain Profile of an individual has occurred, said method comprising:
a. providing means for the computer system to compute the individual's Normal Brain Profile, wherein said means for computing the individual's Normal Brain Profile comprises:
i. providing means for the individual to activate and take a test session on the computer system, wherein the test session comprises at least 2 test modules, wherein the at least 2 test modules differ from one another, wherein the at least 2 test modules are each designed to collect certain brain performance data from the individual, wherein the collected brain performance data from the at least 2 test modules correlates to at least one of the individual's neuromotor or cognitive functions, and wherein the computer system computes a separate test module score for each of the particular test session's at least 2 test modules,
ii. providing means for the computer system to compute a Brain Profile Score for this particular test session, wherein the computation of said Brain Profile Score comprises the computer system performing a mathematical manipulation of all of that test session's computed test module scores,
iii. providing means for repeatedly performing steps a.i. and a.ii. at least one additional time, and
iv. providing means for the computer system to compute the individual's Normal Brain Profile, wherein the computation of said Normal Brain Profile comprises the computer system performing a mathematical manipulation of at least 2 of the individual's earlier-computed Brain Profile Scores;
b. providing means for the computer system to compute the individual's Current Brain Profile, wherein said means for computing the individual's Current Brain Profile comprises:
i. providing means for the individual to activate and take a subsequent test session on the computer system, wherein the subsequent test session is any test session taken after the individual's Normal Brain Profile has been computed, and wherein the subsequent test session comprises the at least 2 test modules included in the test session employed when computing the individual's Normal Brain Profile, and
ii. providing means for the computer system to compute the individual's Current Brain Profile Score for this particular subsequent test session, wherein the computation of said Current Brain Profile Score comprises the computer system performing a mathematical manipulation of all of this subsequent test session's computed test module scores;
c. providing means for the computer system to compute the difference between the individual's Normal Brain Profile and the individual's Current Brain Profile;
d. providing means for the computer system to compute whether the difference between the individual's Normal and Current Brain Profiles constitutes a change in the individual's Brain Profile; and
e. providing means for the computer system to report whether a change in the individual's Brain Profile has occurred.

2. The computer-based identifying, monitoring, and reporting method of claim 1, wherein said means for computing the individual's Normal Brain Profile further comprises:
a. providing means for the computer system to characterize each computed Brain Profile Score as either “Qualifying” or “Non-Qualifying”;
b. providing means for steps a.i. and a.ii. of claim 1 to be repeated until at least 2 Qualifying Brain Profile Scores have been computed; and
c. providing means for the computer system to compute the individual's Normal Brain Profile Score from all of the computed Qualifying Brain Profile Scores.

3. The computer-based identifying, monitoring, and reporting method of claim 2, wherein said means for computing the individual's Normal Brain Profile further comprises:
a. providing means for the computer system to characterize each computed test module score as either “Qualifying” or “Non-Qualifying”; and
b. providing means for the computer system to characterize a Brain Profile Score as “Qualifying” only when each test module score of a particular test session has been characterized as “Qualifying”, and by performing a mathematical manipulation of only those “Qualifying” test module scores.

4. The computer-based identifying, monitoring, and reporting method of claim 3, wherein a test module score is characterized as “Qualifying” only when the individual's brain performance data collected from the corresponding test module resulting in that score falls within a predetermined set of parameters, wherein a predetermined set of parameters is set for each brain function being tested by that particular test module, wherein each predetermined set parameters comprises an upper and lower limit for the particular brain function being tested by that test module, and wherein each of the upper and lower limits is a multiplicative factor of a human capacity limit of the brain function being tested.

5. The computer-based identifying, monitoring, and reporting method of claim 1, wherein said means for computing the individual's Normal Brain Profile comprises:
a. providing means for the individual to activate and take a test session on the computer system, wherein the test session comprises at least 2 test modules, wherein the at least 2 test modules differ from one another, wherein the at least 2 test modules are each designed to collect certain brain performance data from the individual, wherein the collected brain performance data from each of the at least 2 test modules correlates to at least one of the individual's neuromotor or cognitive functions, and wherein the computer system computes a separate test module score for each of the particular test session's at least 2 test modules;
b. providing means for repeatedly performing step a. at least one additional time;
c. providing means for the computer system to compute a Normal Test Module Score for each of the test session's test modules, wherein the computation of each of said Normal Test Module Score comprises the computer system performing a mathematical manipulation of each particular module's earlier-computed test module scores; and
d. providing means for the computer system to compute the individual's Normal Brain Profile, wherein the computation of said Normal Brain Profile comprises the computer system performing a mathematical manipulation of all of the earlier-computed Normal Test Module Scores.

6. The computer-based identifying, monitoring, and reporting method of claim 5, wherein said means for computing the individual's Current Brain Profile comprises:
a. providing means for the individual to activate and take a subsequent test session on the computer system, wherein the subsequent test session comprises the at least 2 test modules employed when computing the individual's Normal Brain Profile, and wherein the computer system computes a separate current test module score for each of the particular subsequent test session's at least 2 test modules;
b. providing means for the computer system to compute a Test Module Differential for each of the particular subsequent test session's at least 2 test modules, wherein the computation of each of said Test Module Differential comprises the computer system performing a mathematical manipulation of each of this subsequent test session's at least 2 test module's current test module score and that particular test module's corresponding and earlier-computed Normal Test Module Score; and
c. providing means for the computer system to compute the individual's Current Brain Profile, wherein the computation of said Current Brain Profile comprises the computer system performing a mathematical manipulation of all of the earlier-computed Test Module Differentials.

7. The computer-based identifying, monitoring, and reporting method of claim 5, wherein said means for computing the individual's Normal Brain Profile further comprises:
a. providing means for the computer system to characterize each computed Brain Profile Score as “Qualifying” or “Non-Qualifying”;
b. providing means for steps a.i. and a.ii. of claim 1 to be repeated until at least 2 Qualifying Brain Profile Scores have been computed; and
c. providing means for the computer system to compute the individual's Normal Brain Profile Score from all of the computed Qualifying Brain Profile Scores.

8. The computer-based identifying, monitoring, and reporting method of claim 7, wherein said means for computing the individual's Normal Brain Profile further comprises:
a. providing means for the computer system to characterize each computed test module score as either “Qualifying” or “Non-Qualifying”; and
b. providing means for the computer system to compute Qualifying Brain Profile Scores only when each test module score of a particular test session has been characterized as “Qualifying”, and by performing a mathematical manipulation of only those “Qualifying” test module scores.

9. The computer-based identifying and reporting method of claim 8, wherein a test module score is characterized as “Qualifying” only when the individual's brain performance data collected from the corresponding test module resulting in that score falls within a predetermined set of parameters, wherein a predetermined set of parameters is set for each brain function being tested by that particular test module, wherein each predetermined set parameters comprises an upper and lower limit for the particular brain function being tested by that test module, and wherein each of the upper and lower limits is a multiplicative factor of a human capacity limit of the brain function being tested.

10. A computer readable medium including instructions for identifying, monitoring, and reporting, on a computer system, whether a change to the Brain Profile of an individual has occurred, said method comprising:
a. instructions for the computer system to compute the individual's Normal Brain Profile, wherein said means for computing the individual's Normal Brain Profile comprises:
i. instructions for the individual to activate and take a test session on the computer system, wherein the test session comprises at least 2 test modules, wherein the at least 2 test modules differ from one another, wherein the at least 2 test modules are each designed to collect certain brain performance data from the individual, wherein the collected brain performance data from the at least 2 test modules correlates to at least one of the individual's neuromotor or cognitive functions, and wherein the computer system computes a separate test module score for each of the particular test session's at least 2 test modules,
ii. instructions for the computer system to compute a Brain Profile Score for this particular test session, wherein the computation of said Brain Profile Score comprises the computer system performing a mathematical manipulation of all of that test session's computed test module scores,
iii. instructions for repeatedly performing steps a.i. and a.ii. at least one additional time, and
iv. instructions for the computer system to compute the individual's Normal Brain Profile, wherein the computation of said Normal Brain Profile comprises the computer system performing a mathematical manipulation of at least 2 of the individual's earlier-computed Brain Profile Scores;
b. instructions for the computer system to compute the individual's Current Brain Profile, wherein said means for computing the individual's Current Brain Profile comprises:
i. instructions for the individual to activate and take a subsequent test session on the computer system, wherein the subsequent test session is any test session taken after the individual's Normal Brain Profile has been computed, and wherein the subsequent test session comprises the at least 2 test modules included in the test session employed when computing the individual's Normal Brain Profile, and
ii. instructions for the computer system to compute the individual's Current Brain Profile Score for this particular subsequent test session, wherein the computation of said Current Brain Profile Score comprises the computer system performing a mathematical manipulation of all of this subsequent test session's computed test module scores;
c. instructions for the computer system to compute the difference between the individual's Normal Brain Profile and the individual's Current Brain Profile;
d. instructions for the computer system to compute whether the difference between the individual's Normal and Current Brain Profiles constitutes a change in the individual's Brain Profile; and
e. instructions for instructions for the computer system to report whether a change in the individual's Brain Profile has occurred.

11. The computer readable medium of claim 10, wherein said means for computing the individual's Normal Brain Profile further comprises:
a. instructions for the computer system to characterize each computed Brain Profile Score as either “Qualifying” or “Non-Qualifying”;
b. instructions for steps a.i. and a.ii. of claim 10 to be repeated until at least 2 Qualifying Brain Profile Scores have been computed; and
c. instructions for the computer system to compute the individual's Normal Brain Profile Score from all of the computed Qualifying Brain Profile Scores.

12. The computer readable medium of claim 11, wherein said means for computing the individual's Normal Brain Profile further comprises:
a. instructions for the computer system to characterize each computed test module score as either “Qualifying” or “Non-Qualifying”; and
b. instructions for the computer system to providing to characterize a Brain Profile Score as “Qualifying” only when each test module score of a particular test session has been characterized as “Qualifying”, and by performing a mathematical manipulation of only those “Qualifying” test module scores.

13. The computer readable medium of claim 10, wherein said means for computing the individual's Normal Brain Profile comprises:
a. instructions for the individual to activate and take a test session on the computer system, wherein the test session comprises at least 2 test modules, wherein the at least 2 test modules differ from one another, wherein the at least 2 test modules are each designed to collect certain brain performance data from the individual, wherein the collected brain performance data from each of the at least 2 test modules correlates to at least one of the individual's neuromotor or cognitive functions, and wherein the computer system computes a separate test module score for each of the particular test session's at least 2 test modules;
b. instructions for repeatedly performing step a. at least one additional time;
c. instructions for the computer system to compute a Normal Test Module Score for each of the test session's test modules, wherein the computation of each of said Normal Test Module Score comprises the computer system performing a mathematical manipulation of each particular module's earlier-computed test module scores; and
d. instructions for the computer system to compute the individual's Normal Brain Profile, wherein the computation of said Normal Brain Profile comprises the computer system performing a mathematical manipulation of all of the earlier-computed Normal Test Module Scores.

14. The computer readable medium of claim 13, wherein said means for computing the individual's Current Brain Profile comprises:
a. instructions for the individual to activate and take a subsequent test session on the computer system, wherein the subsequent test session comprises the at least 2 test modules employed when computing the individual's Normal Brain Profile, and wherein the computer system computes a separate current test module score for each of the particular subsequent test session's at least 2 test modules;
b. instructions for the computer system to compute a Test Module Differential for each of the particular subsequent test session's at least 2 test modules, wherein the computation of each of said Test Module Differential comprises the computer system performing a mathematical manipulation of each of this subsequent test session's at least 2 test module's current test module score and that particular test module's corresponding and earlier-computed Normal Test Module Score; and
c. instructions for the computer system to compute the individual's Current Brain Profile, wherein the computation of said Current Brain Profile comprises the computer system performing a mathematical manipulation of all of the earlier-computed Test Module Differentials.

15. A computer-based method for delivering computer-readable instructions for identifying and reporting, on a computer system, whether a change to the Brain Profile of an individual has occurred, said method comprising:
a. transmitting, over a signal transmission medium, signals representative of instructions for the computer system to compute the individual's Normal Brain Profile, wherein said means for computing the individual's Normal Brain Profile comprises:
i. transmitting, over a signal transmission medium, signals representative of instructions for the individual to activate and take a test session on the computer system, wherein the test session comprises at least 2 test modules, wherein the at least 2 test modules differ from one another, wherein the at least 2 test modules are each designed to collect certain brain performance data from the individual, wherein the collected brain performance data from the at least 2 test modules correlates to at least one of the individual's neuromotor or cognitive functions, and wherein the computer system computes a separate test module score for each of the particular test session's at least 2 test modules,
ii. transmitting, over a signal transmission medium, signals representative of instructions for the computer system to compute a Brain Profile Score for this particular test session, wherein the computation of said Brain Profile Score comprises the computer system performing a mathematical manipulation of all of that test session's computed test module scores,
iii. transmitting, over a signal transmission medium, signals representative of instructions for repeatedly performing steps a.i. and a.ii. at least one additional time, and
iv. transmitting, over a signal transmission medium, signals representative of instructions for the computer system to compute the individual's Normal Brain Profile, wherein the computation of said Normal Brain Profile comprises the computer system performing a mathematical manipulation of at least 2 of the individual's earlier-computed Brain Profile Scores;
b. transmitting, over a signal transmission medium, signals representative of instructions for the computer system to compute the individual's Current Brain Profile, wherein said means for computing the individual's Current Brain Profile comprises:
i. transmitting, over a signal transmission medium, signals representative of instructions for the individual to activate and take a subsequent test session on the computer system, wherein the subsequent test session is any test session taken after the individual's Normal Brain Profile has been computed, and wherein the subsequent test session comprises the at least 2 test modules included in the test session employed when computing the individual's Normal Brain Profile, and
ii. transmitting, over a signal transmission medium, signals representative of instructions for the computer system to compute the individual's Current Brain Profile Score for this particular subsequent test session, wherein the computation of said Current Brain Profile Score comprises the computer system performing a mathematical manipulation of all of this subsequent test session's computed test module scores;
c. transmitting, over a signal transmission medium, signals representative of instructions for the computer system to compute the difference between the individual's Normal Brain Profile and the individual's Current Brain Profile;
d. transmitting, over a signal transmission medium, signals representative of instructions for the computer system to compute whether the difference between the individual's Normal and Current Brain Profiles constitutes a change in the individual's Brain Profile; and
e. transmitting, over a signal transmission medium, signals representative of instructions for the computer system to report whether a change in the individual's Brain Profile has occurred.

16. The computer-based method for delivering computer-readable instructions of claim 15, wherein said means for computing the individual's Normal Brain Profile further comprises:
a. transmitting, over a signal transmission medium, signals representative of instructions for the computer system to characterize each computed Brain Profile Score as either “Qualifying” or “Non-Qualifying”;
b. transmitting, over a signal transmission medium, signals representative of instructions for steps a.i. and a.ii. of claim 15 to be repeated until at least 2 Qualifying Brain Profile Scores have been computed; and
c. transmitting, over a signal transmission medium, signals representative of instructions for the computer system to compute the individual's Normal Brain Profile Score from all of the computed Qualifying Brain Profile Scores.

17. The computer-based method for delivering computer-readable instructions of claim 16, wherein said means for computing the individual's Normal Brain Profile further comprises:
a. transmitting, over a signal transmission medium, signals representative of instructions for the computer system to characterize each computed test module score as either “Qualifying” or “Non-Qualifying”; and
b. transmitting, over a signal transmission medium, signals representative of instructions for the computer system to compute Qualifying Brain Profile Scores only when each test module score of a particular test session has been characterized as “Qualifying”, and by performing a mathematical manipulation of only those “Qualifying” test module scores.

18. The computer-based method for delivering computer-readable instructions of claim 15, wherein said means for computing the individual's Normal Brain Profile comprises:
a. transmitting, over a signal transmission medium, signals representative of instructions for the individual to activate and take a test session on the computer system, wherein the test session comprises at least 2 test modules, wherein the at least 2 test modules differ from one another, wherein the at least 2 test modules are each designed to collect certain brain performance data from the individual, wherein the collected brain performance data from each of the at least 2 test modules correlates to at least one of the individual's neuromotor or cognitive functions, and wherein the computer system computes a separate test module score for each of the particular test session's at least 2 test modules;
b. transmitting, over a signal transmission medium, signals representative of instructions for repeatedly performing step a. at least one additional time;
c. transmitting, over a signal transmission medium, signals representative of instructions for the computer system to compute a Normal Test Module Score for each of the test session's test modules, wherein the computation of each of said Normal Test Module Score comprises the computer system performing a mathematical manipulation of each particular module's earlier-computed test module scores; and
d. transmitting, over a signal transmission medium, signals representative of instructions for the computer system to compute the individual's Normal Brain Profile, wherein the computation of said Normal Brain Profile comprises the computer system performing a mathematical manipulation of all of the earlier-computed Normal Test Module Scores.

19. The computer-based method for delivering computer-readable instructions of claim 18, wherein said means for computing the individual's Current Brain Profile comprises:
a. transmitting, over a signal transmission medium, signals representative of instructions for the individual to activate and take a subsequent test session on the computer system, wherein the subsequent test session comprises the at least 2 test modules employed when computing the individual's Normal Brain Profile, and wherein the computer system computes a separate current test module score for each of the particular subsequent test session's at least 2 test modules;
b. transmitting, over a signal transmission medium, signals representative of instructions for the computer system to compute a Test Module Differential for each of the particular subsequent test session's at least 2 test modules, wherein the computation of each of said Test Module Differential comprises the computer system performing a mathematical manipulation of each of this subsequent test session's at least 2 test module's current test module score and that particular test module's corresponding and earlier-computed Normal Test Module Score; and
c. transmitting, over a signal transmission medium, signals representative of instructions for the computer system to compute the individual's Current Brain Profile, wherein the computation of said Current Brain Profile comprises the computer system performing a mathematical manipulation of all of the earlier-computed Test Module Differentials.
